(19) United States
(12) Patent Application Publication
(10) Pub. No.: US 2005/0215478 A1
(43) Pub. Date: Sep. 29, 2005

(54) Amino acid sequences capable of facilitating penetration across a biological barrier

(76) Inventors: Shmuel A. Ben-Sasson, Jerusalem (IL); Einat Cohen, Jerusalem (IL)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/501,838

(22) PCT Filed: Feb. 7, 2003
(86) PCT No.: PCT/IB03/00968

§ 371 Date: Nov. 29, 2004

Related U.S. Application Data
(60) Provisional application No. 60/355,396, filed on Feb. 7, 2002.

(51) Int. Cl. A61K 38/10; A61K 38/08; C07K 7/08

(52) U.S. Cl. 514/13; 514/14; 514/15; 530/326; 530/327; 530/328

(57) ABSTRACT

The invention relates to amino acid sequences capable of facilitating penetration of an effector across a biological barrier. The invention also relates to methods of treating preventing diseases by administering or penetrating modules to affected subjects.

### TECHNICAL FIELD OF THE INVENTION

This invention relates to amino acid sequences capable of facilitating penetration of an effector across biological barriers.

### BACKGROUND OF THE INVENTION

Techniques enabling efficient transfer of a substance of interest across a biological barrier are of considerable interest in the field of biotechnology. For example, such techniques may be used for the transport of a variety of different substances across a biological barrier regulated by tight junctions (i.e., the mucosal epithelia, which includes the intestinal and respiratory epithelia and the vascular endothelia, which includes the blood-brain barrier).

The intestinal epithelium represents the major barrier to absorption of orally administered compounds, e.g., drugs and peptides, into the systemic circulation. This barrier is composed of a single layer of columnar epithelial cells (primarily enterocytes, goblet cells, endocrine cells, and paneth cells), which are joined at their apical surfaces by the tight junctions. See Madara et al., PHYSIOLOGY OF THE GASTROINTESTINAL TRACT; 2^{nd} Ed., Johnson, ed., Raven Press, New York, pp. 1251-66 (1987).

Compounds that are presented in the intestinal lumen can enter the blood stream through active or facilitative transport, passive transcellular transport, or passive paracellular transport. Active or facilitative transport occurs via cellular carriers, and is limited to transport of low molecular weight degradation products of complex molecules such as proteins and sugars, e.g., amino acids, pentoses, and hexoses. Passive transcellular transport requires partitioning of the molecule through both the apical and basolateral membranes. This process is limited to relatively small hydrophobic compounds. See Jackson, PHYSIOLOGY OF THE GASTROINTESTINAL TRACT 2^{nd} Ed., Johnson, ed., Raven Press, New York, pp. 1597-1621 (1987). Consequently, with the exception of those molecules that are transported by active or facilitative mechanisms, absorption of larger, more hydrophilic molecules is, for the most part, limited to the paracellular pathway. However, the entry of molecules through the paracellular pathway is primarily restricted by the presence of the tight junctions. See Gumbiner, Am. J. Physiol., 253:C749-C758 (1987); Madara, J. Clin. Invest., 83:1089-94 (1989).

In transmission electron microscopy, tight junctions appear as an approximately 80 nm long region at the boundary of neighboring cells in which the plasma membranes of adjacent cells are brought into close opposition. See Farquhar, et al., J. Cell Biol., 17:375-412 (1963). This structure circumscribes epithelial cells immediately below the brush border (apical domain), thereby forming a seal between epithelial cells and their neighbors. See Pappenheimer, et al., J. Membrane Biol., 102:2125-2136 (1986); Claude et al., J. Cell Biol., 58:390-400 (1973); and Bakker, et al., J. Membrane Biol., 98:1209-1221 (1985). The tight junctions function to define apical and basal membrane polarity by limiting the exchange of membrane lipids and proteins between the two and to regulate the paracellular transport of water, solutes, and immune cells. See Heiskala, et al., Traffic, 2:92-98 (2001).

Considerable attention has been directed to finding ways to increase paracellular transport by “loosening” tight junctions. One approach to overcoming the restriction to paracellular transport is to co-administer, in a mixture, biologically active ingredients with absorption enhancing agents. Generally, intestinal/respiratory absorption enhancers include, but are not limited to, calcium chelators, such as citrate and ethylenediamine tetraacetic acid (EDTA); surfactants, such as sodium dodecyl sulfate, bile salts, palmitoylcamitine, and sodium salts of fatty acids; or toxins, such as zonula occludens toxin (“ZOT”). ZOT functions by increasing the bioavailability of oral insulin in diabetic animals. See Fasano and Uzzau, J. Clin. Invest., 99:1158-64 (1997). EDTA, which is known to disrupt tight junctions by chelating calcium, enhances the efficiency of gene transfer into the airway respiratory epithelium in patients with cystic fibrosis. See Wang, et al., Am. J. Respir. Cell Mol. Biol., 22:129-138 (2000). However, one drawback to all of these methods is that they facilitate the indiscriminate penetration of any nearby molecule that happens to be in the gastrointestinal or airway lumen. In addition, each of these intestinal/respiratory absorption enhancers has properties that limit their general usefulness as a means to promote absorption of various molecules across a biological barrier.

Moreover, with the use of surfactants, the potential lytic nature of these agents raises concerns regarding safety. Specifically, the intestinal and respiratory epithelia provides a barrier to the entry of toxins, bacteria and viruses from the hostile exterior. Hence, the possibility of exfoliation of the epithelium using surfactants, as well as the potential complications arising from increased epithelial repair, raise safety concerns about the use of surfactants as intestinal/respiratory absorption enhancers.

When calcium chelators are used as intestinal/respiratory absorption enhancers, Ca⁺² depletion does not act directly on the tight junction, but, rather, induces global changes in the cells, including disruption of actin filaments, disruption of adherent junctions, diminished cell adhesion, and activation of protein kinases. See Citi, J. Cell Biol., 117:169-178 (1992). Moreover, as typical calcium chelators only have access to the mucosal surface, and luminal Ca⁺² concentration may vary, sufficient amounts of chelators generally cannot be administered to lower Ca⁺² levels to induce the opening of tight junctions in a rapid, reversible, and reproducible manner.

Additionally, some toxins such as Clostridium difficile toxin A and B, appear to irreversibly increase paracellular permeability and are thus, associated with destruction of the tight junction complex. See Hecht, et al., J. Clin. Invest., 82:1516-24 (1988); Fiorentini and Thelestam, Toxicon, 29:543-67 (1991). Other toxins such as Vibrio cholerae zonula occludens toxin (ZOT) modulate the structure of intercellular tight junctions. As a result, the intestinal mucosa becomes more permeable. See Fasano, et al., Proc. Nat. Acad Sci., USA, 8:524246 (1991); U.S. Pat. No. 5,827,534. However, this also results in diarrhea.

Thus, a need remains for an efficient, specific, non-invasive, low-risk means to target various biological barriers for the delivery of biologically active molecules such as polypeptides, drugs and other therapeutic agents.

### SUMMARY OF THE INVENTION

The present invention provides penetrating peptides, which are capable of translocating across a biological barrier. The invention also relates to methods of using such penetrating peptides to translocate an effector across a biological barrier.

In one aspect, the invention involves a penetrating peptide having at least one amino acid sequence selected from: (BX)₄Z(BX)₂ZXB; ZBXB₂XBXB₂XBX₃BXB₂X₂B₂; ZBZX₂B₄XB₃ZXB₄Z₂B₂; ZB₉XBX₂B₂ZBXZBX₂; BZB₈XB₉X₂ZXB; B₂ZXZB₅XB₂XB₂X₂BZXB₂; XB₉XBXB₆X₃B; X₂B₃XB₄ZBXB₄XBₙXB; XB₂XZBXZB₂ZXBX₃BZXBX₃B; BZXBXZX₂B₄XBX₂B₂XB₄X₂; BZXBXZX₂B₄XBX₂B₂XB₄; B₂XZ₂XB₄XBX₂B₅X₂B₂; B_{q}XₜZBₘX_{q}B₄XBXₙBₘZB₂X₂B₂; B₂ZX₃ZBₘX_{q}B₄XBXₙBₘZB₂X₂B₂; X₃ZB₆XBX₃BZB₂X₂B₂; and at least 12 contiguous amino acids of any of these amino acid sequences, where X is any amino acid; B is a hydrophobic amino acid belonging to the group consisting of A, V, L, L M, F and P (single letter amino acid codes); and Z is a charged amino acid belonging to the group consisting of K, R, D and E; and where q is 0 or 1; m is 1 or 2; and n is 2 or 3; and where t is 1 or 2 or 3; and where the penetrating peptide is capable of translocating across a biological barrier.

In one embodiment, the invention provides a penetrating peptide having an amino acid sequence of any one of SEQ ID NOS: 1-15 and 24-29. In addition, the penetrating peptides of the invention include peptides having at least 12 contiguous amino acids of any of the peptides defined by SEQ ID NOS: 1-15 and 24-29. In various embodiments, the penetrating peptide can be less than thirty (30), less than twenty-five (25), or less than twenty (20) amino acids in length. The invention also includes mutant or variant peptides any of whose residues may be changed from the corresponding residues shown in SEQ ID NOS: 1, 2, 3, 4, 5, 6, 7, 8, 9, 10, 11, 12, 13, 14, 15, 24, 25, 26, 27, 28 or 29, while still encoding a peptide that maintains its penetrating activities and physiological functions, or functional fragments thereof In one embodiment, the fragment of an amino acid sequence of any one of SEQ ID NOS: 1-15 and 24-29 is at least 10 amino acids in length. The amino acid sequence of the penetrating peptide variant may contain a conservative or a non-conservative amino acid substitution.

In general, a penetrating peptide variant that preserves the translocating function includes any variant in which residues at a particular position in the sequence have been substituted by other amino acids, and further includes the possibility of inserting an additional residue or residues between two residues of the parent protein as well as the possibility of deleting one or more residues from the parent sequence. Any such amino acid substitution, insertion, or deletion is encompassed by the invention. In favorable circumstances, the substitution is a conservative substitution.

In some embodiments, amino acid substitutions at “non-essential” amino acid residues can be made in the penetrating peptides. A “non-essential” amino acid residue is a residue that can be altered from the native sequences of the penetrating peptides without altering their biological activity, whereas an “essential” amino acid residue is required for such biological activity. For example, amino acid residues that are conserved among the penetrating peptides of the invention are predicted to be particularly non-amenable to substantial alteration. Amino acids for which conservative substitutions can be made are well known within the art.

Mutations can be introduced into nucleic acids encoding penetrating peptides by standard techniques, including, but not limited to site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the penetrating peptide is replaced with another amino acid residue from the same side chain family. Alternatively, in another embodiment, mutations can be introduced randomly along all or part of a penetrating peptide coding sequence, such as by saturation mutagenesis, and the resultant mutants can be screened for biological activity to identify mutants that retain activity. Following mutagenesis, the encoded penetrating peptide can be expressed by any recombinant technology known in the art and the activity of the protein can be determined. Amino acid substitutions can also be introduced during artificial peptide synthesis such as solid-phase synthesis of peptides.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NREQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

The invention also includes analogs in which one or more peptide bonds have been replaced with an alternative type of covalent bond (a “peptide mimetic”) that is not susceptible to cleavage by peptidases elaborated by the subject. Where proteolytic degradation of a peptide composition is encountered following administration to the subject, replacement of a particularly sensitive peptide bond with a noncleavable peptide mimetic renders the resulting peptide derivative compound more stable and thus more useful as a therapeutic. Such mimetics, and methods of incorporating them into peptides, are well known in the art.

Similarly, the replacement of an L-amino acid residue by a D-amino acid residue is a standard method for rendering the compound less sensitive to enzymatic destruction. Other amino acid analogs are known in the art, such as norleucine, norvaline, homocysteine, homoserine, ethionine, and the like. Also useful is derivatizing the compound with an amino-terminal blocking group such as a t-butyloxycarbonyl, acetyl, methyl, succinyl, methoxysuccinyl, suberyl, adipyl azelayl, dansyl, benzyloxycarbonyl, fluorenylmethoxycarbonyl, methoxyaselayl, methoxyadipyl, methoxysuberyl, and a 2,3-dinitrophenyl group.

The invention also includes the replacement of the N-terminal amino acid in said sequences by methionine or the addition of between 1 to 4 amino acids, starting with methionine, at the N-terminus of the sequences in order to enable the biosynthesis of recombinant constructs of the sequences using the canonical start codon of methionine, which is well-known in the art.

In some embodiments, the penetrating peptide of the invention can be further chemically modified. For example, one or more polyethylene glycol (PEG) residues can be attached to the penetrating peptides of the invention.

As used herein, a “penetrating peptide” is any peptide that facilitates the translocation of a substance across a biological barrier. Examples of biological barriers include, but are not limited to, tight junctions and the plasma membrane. Moreover, those skilled in the art will recognize that translocation may occur across a biological barrier in a tissue such as epithelial cells or endothelial cells.

In some embodiments, the invention involves a penetrating module that is a penetrating peptide of the invention fused, coupled, or attached to an effector. The “effector” can be any suitable molecule, including, but not limited to DNA, RNA, a protein, a peptide, or a pharmaceutically active agent, such as, for example, a hormone, a growth factor, a neurotrophic factor, a bioactive peptide, heparin, a toxin, an antibiotic, an antipathogenic agent, an antigen, an antibody, an antibody fragment, an immunomodulator, and an enzyme; or a therapeutic agent. Bioactive peptides include, for example, insulin, growth hormone, a gonadotropin, a growth factor, erythropoietin, granulocyte/monocyte colony stimulating factor (GM-CSF), αMSH, enkephalin, dalargin, kyotorphin, EPO, bFGF, hirulog, a lutenizing hormone releasing hormone (LHRH) analog, and neurotrophic factors. Pharmaceutically active agents include, for example, an anticoagulant, a toxin, an antibiotic, an antipathogenic agent, an antigen, an antibody fragment, a vitamin, an immunomodulator, an enzyme, an antineoplastic agent, heparin, methotraxate, and a therapeutic agent.

As used herein, the terms “fusion” or “fused” or “coupled” or “attached” are meant to include all such specific interactions that result in two or more molecules showing a preference for one another relative to some third molecule including any type of interaction enabling a physical association between an effector or a molecular vessel and a penetrating peptide. This includes, but is not limited to, processes such as covalent, ionic, hydrophobic, and hydrogen bonding, but does not include non-specific associations such as solvent preferences. The association must be sufficiently strong so that the effector does not disassociate before or during penetration of the biological barrier. Fusion may be achieved using any chemical, biochemical, enzymatic or genetic coupling method known to those skilled in the art. The effector of interest is preferably coupled to the C-terminal end of the penetrating peptide.

In other embodiments, the penetrating peptide could also be a complex consisting of the penetrating peptide attached to a “molecular vessel” in which an effector is enclosed. Suitable effectors include, but are not limited to, any of the suitable bioactive peptides or pharmaceutically active agents described herein. A molecular vessel could be a soluble receptor, or part of a soluble receptor (e.g., a “minireceptor”, as described in Kristensens, et al., J. Biol. Chem., 274(52):37351-56 (1999)). A molecular vessel can also be a binding protein. The molecular vessel will serve as a high affinity binding pocket for the delivery of the intact effector, such as a hormone, a growth factor, or any other effector. One example of a molecular vessel is a soluble insulin receptor, or the ligand-binding domain of the insulin receptor (e.g., the above-mentioned “minireceptor”), to bind and deliver insulin as an effector. Another example for the use of a molecular vessel to deliver non-permeable effectors, is the use of Intrinsic factor, attached to the penetrating peptide, in order to enable the delivery of vitamin B12 as an effector. In a way, the molecular vessel serves as a “Trojan horse” to translocate the otherwise impermeable effector across a biological barrier. The “effector” can be any suitable molecule as defined above.

Another embodiment of the invention involves a method of bulk translocation of a pharmaceutical composition. The peptides described herein serve as the basis for the design of therapeutic drug-containing micro particles/droplets. For example, penetrating peptides are attached to fatty moieties and incorporated at the interface of a hydrophobic vesicle which contains the desired pharmaceutical composition. This can be achieved via amidation of the free amino group of extra lysine(s), added at the C-terminus of the penetrating peptide, using long fatty acids such as stearoyl, palmitoyl or myristoyl.

Another embodiment of the invention involves a method of oral vaccination by administering to a subject a penetrating peptide of the invention attached to a desirable antigenic protein.

In one embodiment, the invention involves a method of translocating a penetrating peptide of the invention across a biological barrier. In another embodiment, the invention involves methods of translocating an effector across a biological barrier by coupling the effector to a penetrating peptide module that can be introduced to the biological barrier.

As used herein, the term “biological barrier” is meant to include biological membranes such as the plasma membrane as well as any biological structures sealed by tight junctions (or occluding junctions) such as the mucosal epithelia, including, but not limited to, the intestinal or respiratory epithelia or the vascular endothelia, including, but not limited to, the blood-brain barrier.

In still further embodiments, the invention includes a pharmaceutical composition containing a therapeutically or prophylactically effective amount of one or more penetrating peptide and a pharmaceutically acceptable carrier.

Preferred “pharmaceutical compositions” include enteric coated tablets and gelatin capsules comprising the active ingredient together with a) diluents, e.g. lactose, dextrose, sucrose, mannitol, sorbitol, cellulose and/or glycine; b) protease inhibitors such as Aprotinin or trasylol; c) lubricants, e.g., silica, talcum, stearic acid, its magnesium or calcium salt, poloxamer and/or polyethyleneglycol; for tablets also d) binders, e.g., magnesium aluminum silicate, starch paste, gelatin, tragacanth, methylcellulose, sodium carboxymethylcellulose and/or polyvinylpyrrolidone; e) ionic surface active agents such as bile salts, if desired f) disintegrants, e.g., starches, agar, alginic acid or its sodium salt, or effervescent mixtures; and/or g) absorbents, colorants, flavors and sweeteners. Injectable compositions are preferably aqueous isotonic solutions or suspensions, and suppositories are advantageously prepared from fatty emulsions or suspensions. The compositions may be sterilized and/or contain adjuvants, such as preserving, reducing agents e.g., NAC (N-Acetyl-L-Cysteine), stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.01 to 75%, preferably about 0.1 to 10%, of the active ingredient.

These compositions may further contain a mixture of at least two substances selected from the group consisting of a non-ionic detergent, an ionic detergent, a protease inhibitor, and a reducing agent. For example, the non-ionic detergent may be a poloxamer; the poloxamer may be pluronic F-68; the ionic detergent may be a bile salt; and the bile salt may be Taurodeoxychilate; the protease inhibitor may be selected from the group consisting of aprotonir and soy bean trypsin inhibitor, and/or the reducing agent may be NAC.

The invention also provides a method for producing a penetrating peptide by coupling an effector to a penetrating peptide. For example, the penetrating peptides can be produced by transfecting a production cell with a vector that has a nucleic acid molecule of a fusion protein encoding the penetrating peptide and an effector operably linked to an expression control sequence; culturing the production cell under conditions that permit production of a fusion protein that includes the penetrating peptide and an effector peptide; and isolating the fusion protein. For example, the penetrating peptides can also be produced by using solid-phase peptides synthesis methods, as is well known in the art. Penetrating peptides that are produced by the methods for producing a penetrating peptide of the invention can also be further chemically modified. For example, one or more polyethylene glycol (PEG) residues can be attached to the penetrating peptides of the invention.

In another embodiment, the invention provides a method of producing a penetrating peptide comprising a penetration peptide and an effector. The effector may be coupled to the penetration peptide by a covalent or a non-covalent bond. For example, the covalent bond may be a peptide bond or the covalent bond may be achieved by a homo- or a hetero-functional bridging reagent. The bridging reagent may be a succinimidyl-(N-maleimidomethyl) cyclohexane-1-carboxylate (SMCC)-type reagent. The covalent bond may also be achieved by using a peptide linker. In some embodiments, the peptide linker may have an amino acid sequence of SEQ ID NO: 16 or 17 and may be cleaved by an enzyme. In some embodiments, the enzyme may be conditionally activated under a certain physiological state, and the released factor may favorably influence that physiological state. In other embodiments, the non-covalent bond may be achieved by an attachment of a hydrophobic moiety to the penetrating peptide, such that the hydrophobic moiety enables the penetrating peptide to be incorporated at the interface of a hydrophobic vesicle in which the effector is contained. In other embodiments, the non-covalent bond may be the result of a biotin-avidin or biotin-streptavidin interaction.

In various other embodiments, the penetrating peptides are derived from a pathogenic or non-pathogenic bacteria, characterized by the ability to penetrate biological barriers in vivo. In some embodiments, the penetrating peptides are derived from integral membrane proteins of pathogenic or non-pathogenic bacteria. In other embodiments, the penetrating peptides are derived from extracellular proteins released by pathogenic or non-pathogenic bacteria. In other embodiments, the penetrating peptide is derived from a bacterial toxin. In still other embodiments, the penetrating peptides are derived from human neurokinin receptors, characterized by the ability to penetrate biological barriers in viva, or are derived from viral proteins.

In still further embodiments, the invention includes a kit having one or more containers containing a therapeutically or prophylactically effective amount of a pharmaceutical composition.

In still further embodiments the concept of a conditionally-activated effector is designed to allow selective release and activation of proteins under conditions where and at sites in which their activity is beneficial.

In this embodiment, the beneficial protein (the effector) is coupled to the penetrating peptide through a cleavable linker peptide. The cleavage site is endogenously recognized and cleaved by an enzyme of the cascade targeted by the effector. The released effector may act as an inhibitory protein or an activator of desired processes.

Finally, another embodiment of the invention involves a method of treating or preventing a disease or pathological condition by administering to a subject in which such treatment or prevention is desired, a pharmaceutical composition in an amount sufficient to treat or prevent the disease or pathological condition. For example, the disease or condition to be treated may include but are not limited to endocrine disorders, including diabetes, infertility, hormone deficiencies and osteoporosis; neurodegenerative disorders, including Alzheimer's disease, Parkinson's disease, and Huntington's disease; cardiovascular disorders, including atherosclerosis, hyper- and hypocoagulable states, coronary disease, and cerebrovascular events; metabolic disorders, including obesity and vitamin deficiencies; haematological disorders; and neoplastic disease.

The details of one or more embodiments of the invention have been set forth in the accompanying description below. Although any methods and materials similar or equivalent to those described herein can be used in the practice or testing of the present invention, the preferred methods and materials are now described. Other features, objects, and advantages of the invention will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this invention belongs. All patents and publications cited in this specification are incorporated by reference.

### BRIEF DESCRIPTION OF THE DRAWINGS

FIG. 1 shows amino acid sequence alignment of ORF HI0638 and its homologs from other pathogenic bacteria

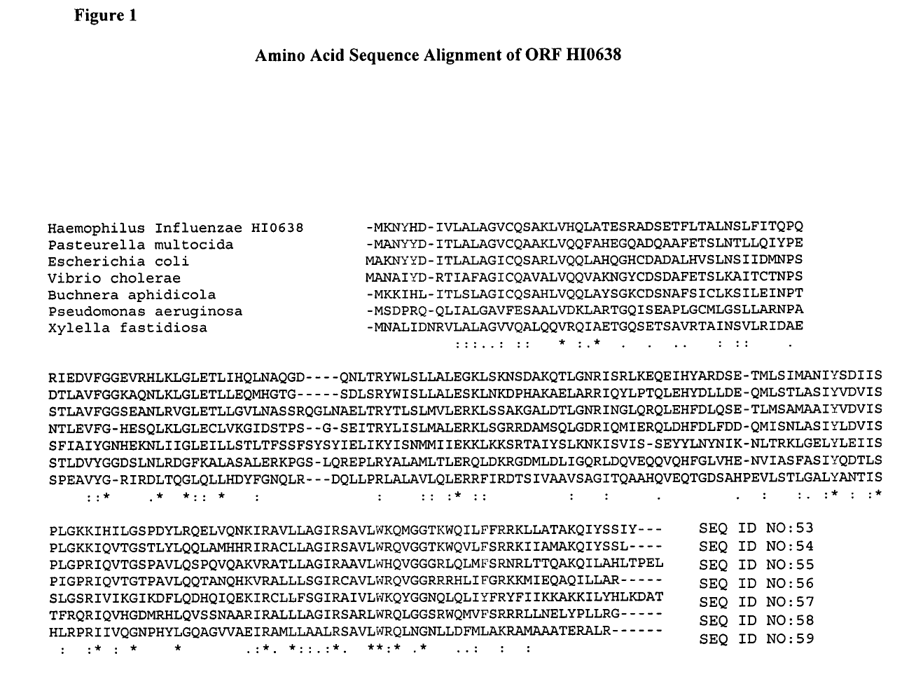

FIG. 2 shows amino acid sequence alignment of the peptides used in this invention as penetrating modules as well as their organism of origin.

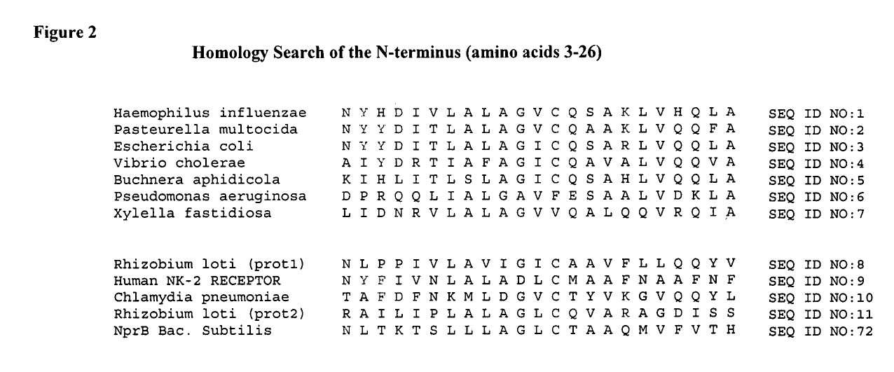

### DETAILED DESCRIPTION OF THE INVENTION

The advantages of the use of small peptide carriers include high quality and purity, low immunogenicity and the potential for highly efficient delivery to biological barriers in an organism. Accordingly, peptide carriers have the potential to improve upon conventional transporters such as liposomes or viruses for the efficient delivery of many macromolecules. The present invention employs a short peptide motif to create a penetrating module to specifically transport macromolecules across biological barriers sealed by tight junctions.

The present invention provides a peptide penetration system that specifically targets various tissues, especially epithelial and endothelial ones, for the delivery of drugs and other therapeutic agents across a biological barrier. Existing transport systems in the art are too limited to be of general application because they are inefficient, alter the biological properties of the active substance, kill the target cell, irreversibly destroy the biological barrier and/or pose too high of a risk to be used in human subjects.

The peptide penetration system of the present invention uses conserved peptide sequences from various proteins involved in paracytosis to create a penetrating module capable of crossing biological barriers. For example, a peptide encoded by or derived from ORF HI0638 of Haemophilus influenzae facilitates penetration of this bacterium between human lung epithelial cells without compromising the epithelial barrier. The peptide sequence encoded by ORF HI0638 is conserved in common pathogenic bacteria or symbiotic including, for example, Haemophilus influenzae, Pasteurella multocida, Escherichia coli, Vibrio cholerae, Buchnera aphidicola, Pseudomonas aeruginosa, and Xylella fastidiosa. A peptide homologous to the N-terminal sequence of HI0638 is also found in other bacteria including, for example, Rhizobium loti, Chlamydia pneumoniae, NprB from Bacillus subtilis, and pilins from Kingella dentrificans and Eikenella corrodens.

Furthermore, a similar peptide sequence is also conserved in proteins of eukaryotic origin such as the neurokinin receptor family proteins, including the human NK-1 and NK-2 receptors. It is known that the neurokinin receptor family is involved in the control of intercellular permeability including plasma extravasation and oedema formation. Extravasation, the leakage and spread of blood or fluid from vessels into the surrounding tissues, often follows inflammatory processes involved in tissue injury, allergy, burns and inflammation. In particular, when NK-1 receptors on blood vessels are activated skin inflammation occurs due to an increase in vascular permeability. See Inoue, et al., Inflamm. Res., 45:316-323 (1996). The neurokinin NK-1 receptor also mediates dural and extracranial plasma protein extravasation, thereby implicating the NK-1 receptor in the pathophysiology of migraine headache. See O'Shaughnessy and Connor, Euro. J. of Pharm., 236:319-321 (1993). The sequences of example penetrating peptides of the invention are shown in Table 1.

TABLE 1

| | | |
|---|---|---|
| Peptide/Organism | Sequence | SEQ ID NO |

| | | |
|---|---|---|
| Peptide 1: from ORF HI0638 | NYHDIVLALAGVCQSAKLVHQLA | (SEQ ID NO:1) |
| Haemophilus influenzae | | |
| Peptide 2: from PM1850 | NYYDITLALAGVCQAAKLVQQFA | (SEQ ID NO:2) |
| Paasteurella multocida | | |
| Peptide 3: from YCFC | NYYDITLALAGICQSARLVQQLA | (SEQ ID NO:3) |
| Escherichia coli | | |
| Peptide 4: from VC1127 | AIYDRTIAFAGICQAVAIVQQVA | (SEQ ID NO:4) |
| Vibrio cholerae | | |
| Peptide 5: from BU262 | KIHLITLSLAGICQSAHLVQQLA | (SEQ ID NO:5) |
| Buchnera aphidicola | | |
| Peptide 6: from PA2627 | DPRQQLIALGAVFESAALVDKLA | (SEQ ID NO:6) |
| Pseudomonas aeruginosa | | |
| Peptide 7: from XF1439 | LIDNRVLALAGVVQALQQVRQIA | (SEQ ID NO:7) |
| Xylella fastidiosa | | |
| Peptide 8: from MLR0187 | NLPPIVLAVIGICAAVFLLQQYV | (SEQ ID NO:8) |
| Rhizobium loti | | |
| Peptide 9: from Human NK-2 | NYFIVNLALADLCMAAFNAAFNF | (SEQ ID NO:9) |
| Receptor | | |
| Peptide 10: from CPN0710/C | TAFDFNKMLDGVCTYVKGVQQYL | (SEQ ID NO:10) |
| Chlamydia pneumoniae | | |
| Peptide 11: from MLR4119 | RAILIPLALAGLCQVARAGDISS | (SEQ ID NO:11) |
| Rhizobium loti | | |
| Peptide 12: from NprB | MRNLTKTSLLLAGLCTAAQMVFVTH | (SEQ ID NO:12) |
| Bacillus subtilis | | |
| Peptide 13: from Pilin | IELMIVIAIIGILAAIALPAYQEYV | (SEQ ID NO:13) |
| Kingella dentrificans | | |
| Peptide 14: from Pilin | IELMIVIAIIGILAAIALPAYQDYV | (SEQ ID NO:14) |
| Eikenella corrodens | | |
| Peptide 15: from zonula | ASFGFCIGRLCVQDGF | (SEQ ID NO:15) |
| occludens toxin (ZOT) | | |
| Peptide 29: from Human NK-1 | NYFLVNLAFAEASMAAFNTVVNF | (SEQ ID NO:24) |
| Receptor | | |
| Peptide 30: from YCFC | MNYYDITLALAGICQSARLVQQLA | (SEQ ID NO:25) |
| Escherichia coli | | |
| Peptide 31: from YCFC | MYYDITLALAGICQSARLVQQLA | (SEQ ID NO:26) |
| Escherichia coil | | |
| Peptide 32: from YCFC | MYDITLALAGICQSARLVQQLA | (SEQ ID NO:27) |
| Escherichia coil | | |
| Peptide 33: from NprB | MRNLTRTSLLLAGLCTAAQMVFV | (SEQ ID NO:28) |
| Bacillus subtilis | | |
| Peptide 34: from ORF HI0638 | NYHDIVLALAGVCQSARLVHQLA | (SEQ ID NO:29) |
| Haemophilus influenrae | | |

The penetrating peptides of the instant invention also include peptides containing at least 12 contiguous amino acids of any of the peptides defined by SEQ ID NOS: 1-15 and 24-29.

The peptide penetration system of the present invention exhibits efficient, non-invasive delivery of an unaltered biologically active substance. For example, the penetrating peptides of the invention can be used in the treatment of diabetes. Insulin levels in the blood stream must be tightly regulated. The penetrating modules of the invention can be used to deliver insulin across the mucosal epithelia at high yield. Alternative non-invasive insulin delivery methods, previously known in the art, have typical yields of 1-5% and cause intolerable fluctuations in the amount of insulin absorbed.

In addition, these penetrating modules or peptides also can be used to treat conditions resulting from atherosclerosis and the formation of thrombi and emboli such as myocardial infarction and cerebrovascular accidents. Specifically, the penetrating modules can be used to deliver heparin across the mucosal epithelia.

The penetrating modules or peptides of the invention also can be used to treat female fertility problems. For example, the penetrating modules or peptides can be used to transport follicle stimulating hormone (“FSH”) across the mucosal epithelia.

Previously, the delivery of effectors (e.g., the delivery of insulin or heparin to the blood stream) required invasive techniques such as intravenous or intramuscular injections. One advantage of the penetrating modules or peptides is that they can deliver effectors across biological barriers through non-invasive administration, including, for example oral, bucal, inhalation, insufflation, transdermal, or depository. In addition, a further advantage of the penetrating modules of the invention is that they can cross the blood-brain barrier, thereby delivering effectors to the CNS.

The peptides described herein serve as the basis for the design of therapeutic “cargos”, namely the coupling of the carriers (“penetrating peptide”) with one or more therapeutic agents (“effectors”). A covalent or noncovalent bond can be used to couple a penetrating peptide to one or more effectors or molecular vessels. Peptide linkers that are cleaved by endogenous peptidases can be used to couple penetrating peptides and effectors or molecular vessels. One example of a detachable peptide linker is the amino acid sequence IEGR (SEQ ID NO: 16) that can be cleaved in the bloodstream by factor Xa (see, e.g., Karlheinz, P., et al., Circulation 101:1158-1164 (2000)).

Peptide linkers are short sequences appearing between the amino acid sequences of the penetrating peptide and the effector. These peptide linkers are cleaved by endogenous peptidases or other enzymes, thereby dissociating the coupling of penetrating peptide to the effector. Such peptide linkers can be utilized for the creation of conditionally activated effectors. Conditionally activated effectors are coupled to penetrating peptides by peptide linkers that are preferentially cleaved during specific physiological processes. Once cleaved, the effector is released to modulate the process—for instance, inhibition of a signal cascade. Preferential cleavage is achieved by using linker sequences cleaved by proteases that are physiologically activated by the triggered cascade. Thus, the conditionally activated effector serves as a “negative feedback” mechanism that inhibits the physiologic process which activated it.

One example of a conditionally activated effector is a thrombolysis activator, e.g., tPA (tissue plasminogen activator). By using the above mentioned linker (i.e., SEQ ID NO. 16) for cleavage by factor Xa, tPA is preferentially released in sites of active coagulation. This way excessive coagulation cascades can be modulated to reduce pathologic thrombosis due to the local release of tPA. Alternatively, other coagulation inhibitors may be used in place of tPA, e.g., hirudin, hirolog, protein C, protein C activator or protein S.

Another example of conditionally activated effectors is a complement cascade inhibitor, e.g., Clq inhibitor. In this example a peptide linker is incorporated that is specifically cleaved by elements of the complement cascade, e.g., C3a Thus, excessive complement activation is inhibited, thereby attenuating the extent of pathologic inflammatory processes.

By way of non-limiting example, conditionally activated effectors according to the invention may be used, e.g., in the inhibition of excessive blood clotting, in the inhibition of pathological inflammatory processes, in the inhibition of high blood pressure and congestive heart failure by angiotensin converting enzyme (ACE)-dependent release of an appropriate effector such as brain-derived natriuric peptide (BNP), or in the inhibition of excessive extracellular-matrix degradation, such as the degradation that occurs during inflammatory processes or tumor invasion, by matrix metaloproteinases (MPP)-dependent release of an effector such as tissue inhibitor of metaloproteinases (TIMP).

Alternatively, the penetrating peptide can be attached to a linker to which imaging compounds can be covalently attached, for example through free amino groups of lysine residues. Such a linker includes, but is not limited to, the amino acid sequence GGKGGK (SEQ ID NO:17).

A penetrating peptide is a peptide that facilitates the passage, translocation, or penetration of a substance across a biological barrier, particularly between cells “sealed” by tight junctions. Translocation may be detected by any method known to those skilled in the art, including using radioactive tagging and/or fluorescent probes incorporated into a penetrating module in conjunction with a paracytosis assay as described in, for example, Schilfgaarde, et al., Infect. and Immun., 68(8):4616-23 (2000). Generally, a paracytosis assay is performed by: a) incubating a cell layer with a penetrating peptide or module; b) making cross sections of the cell layers; and c) detecting the presence of the peptides or peptide modules. The detection step may be carried out by incubating the fixed cell sections with labeled antibodies directed to the peptide, followed by detection of an immunological reaction between the peptide and the labeled antibody. Alternatively, the peptide may be labeled using a radioactive label, or a fluorescent label, or a dye in order to directly detect the presence of the peptide. Further, a bioassay can be used to monitor the peptide translocation. For example, using a bioactive peptide such as insulin, attached to a penetrating module, the drop in blood glucose level can be measured.

As used herein, the term “effector” refers to any molecule or compound of, for example, biological, therapeutic, pharmaceutical, diagnostic, tracing, or food processing interest. It may consist of nucleic acids (ribonucleic acid, deoxyribonucleic acid) from various origins, and particularly of human, viral, animal, eukaryotic or prokaryotic, plant, synthetic origin, etc. A nucleic acid of interest may be of a variety of sizes, ranging from, for example, a simple trace nucleotide to a genome fragment, or an entire genome. It may be a viral genome or a plasmid. Alternatively, the effector of interest may also be a protein, such as, for example, an enzyme, a hormone, a cytokine, an apolipoprotein, a growth factor, a bioactive peptide, an antigen, or an antibody, etc. Furthermore, the effector may be a pharmaceutically active agent, such as, for example, a toxin, a therapeutic agent, or an antipathogenic agent, such as an antibiotic, an antiviral, an antifungal, or an anti-parasitic agent. The effector can also be a physiologically active molecule like a vitamin. The effector of interest may itself be directly active or may be activated in situ by the peptide, by the molecular vessel, by a distinct substance, or by environmental conditions.

The terms “pharmaceutically active agent” and “therapeutic agent” are used herein to refer to a chemical material or compound which, when administered to an organism, induces a detectable pharmacologic and/or physiologic effect.

The penetrating peptide conjugates according to the present invention are characterized by the fact that their penetration capacity is virtually independent of the nature of the effector or molecular vessel that is coupled to it.

Also included in the invention are methods of producing the penetrating peptides and modules described herein. For example, a penetrating peptide or module of the invention can be produced by standard recombinant DNA techniques known in the art. By way of non-limiting example, DNA fragments coding for the different polypeptide sequences can be ligated together in-frame in accordance with conventional techniques, e.g., by employing blunt-ended or stagger-ended termini for ligation, restriction enzyme digestion to provide for appropriate ternini, filling-in of cohesive ends as appropriate, alkaline phosphatase treatment to avoid undesirable joining, and enzymatic ligation. In another embodiment, the fusion gene can be synthesized by conventional techniques including automated DNA synthesizers.

Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, el al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A penetrating peptide-encoding nucleic acid can be cloned into such an expression vector such that the fusion moiety is linked in-frame to the penetrating peptide.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

Recombinant expression vectors comprise a nucleic acid in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. Expression vectors can be introduced into host cells to thereby produce proteins or peptides, including fusion proteins or peptides, encoded by nucleic acids as described herein (e.g., penetrating peptides or modules).

Recombinant expression vectors can be designed for expression of penetrating peptides or modules in prokaryotic or eukaryotic cells. For example, penetrating peptides or modules can be expressed in bacterial cells such as Escherichia coli, insect cells (using baculovirus expression vectors), yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 3140), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET IId (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences encoding the penetrating peptides or modules of the invention can be carried out by standard DNA synthesis techniques.

In another embodiment, the expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, a penetrating peptide or module can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Surmmers, 1989. Virology 170: 31-39).

In yet another embodiment, a nucleic acid encoding the penetrating peptides and modules of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilarnent promoter; Byme and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the a-fetoprotein promoter (Campes and Tilghman, 1989. Genes Dev. 3: 537-546).

The invention further provides a recombinant expression vector comprising a DNA molecule encoding the penetrating peptides and modules of the invention cloned into the expression vector in an antisense orientation. That is, the DNA molecule is operatively-linked to a regulatory sequence in a manner that allows for expression (by transcription of the DNA molecule) of an RNA molecule that is antisense to the penetrating peptide or module mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et aL, “Antisense RNA as a molecular tool for genetic analysis,” Reviews-Trends in Genetics, Vol. 1(1) 1986.

Another aspect of the invention pertains to host cells into which a recombinant expression vector has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the penetrating peptide or module can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “nsfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding the penetrating peptide or module, or can be introduced on a separate vector. Cells stably transfected with the introduced nucleic acid can be identified by drug selection (eg., cells that have incorporated the selectable marker gene will survive, while the other cells die).

A host cell, such as a prokaryotic or eukaryotic host cell in culture, can be used to produce (i.e., express) a penetrating peptide or module. Accordingly, the invention further provides methods for producing penetrating peptides or modules using the host cells. In one embodiment, the method comprises culturing the host cell (into which a recombinant expression vector encoding a penetrating peptide or module has been introduced) in a suitable medium such that the penetrating peptide or module is produced. In another embodiment, the method further comprises isolating the penetrating peptide or module from the medium or the host cell.

The penetrating peptides and modules of the invention can also be produced using solid-phase peptide synthesis methods known in the art. For example, a penetrating peptide can be synthesized using the Merrifield solid-phase synthesis method. (See e.g., Merrifield, R. B., J. Am. Chem. Soc. 85:2149 (1963); ENCYCLOPEDIA OF MOLECULAR BIOLOGY 806 (1st ed. 1994). In this method, the C-terminal amino acid is attached to an insoluble polymeric support resin (e.g., polystyrene beads), thereby forming an immobilized amino acid. To avoid unwanted reactions as the C-terminal amino acid is attached to the resin, the amino group of the C-terminal amino acid is protected or “blocked” using, for example, a tert-butyloxylcarbonyl (t-BOC) group. The blocking group, e.g., t-BOC, on the immobilized amino acid is then removed by adding a dilute acid to the solution. Before a second amino acid is attached to the immobilized peptide chain, the amino-group of the second amino acid is blocked, as described above, and the α-carboxyl group of the second amino acid is activated through a reaction with dicyclohxylcarbdiimide (DCC). The activated a-carboxyl group of the second amino acid then reacts with the free amino group of the immobilized amino acid to form a peptide bond. Additional amino acids are then individually added to the terminal amino acid of the immobilized peptide chain according to the required sequence for the desired penetrating peptide or module. Once the amino acids have been added in the required sequence, the completed peptide is released from the resin, such as for example, by using hydrogen fluoride, which does not attack the peptide bonds.

The penetrating peptides or modules of the invention can also be synthesized using Fmoc solid-phase peptide synthesis. (See e.g., University of Illinois at Urbana-Champaign Protein Sciences Facility, Solid-Phase Peptide Synthesis (SPPS), at http://www.biotech.uiuc.edu/spps.htrn). In this method, the C-terminal amino acid is attached to an insoluble polymeric support resin (e.g., polystyrene beads, cross-linked polystyrene resins, etc.), such as for example, via an acid labile bond with a linker molecule. To avoid unwanted reactions as the C-terminal amino acid is being attached to the resin, the amino group of the C-terminal amino acid is blocked using an Fmoc group. The blocking group, e.g., Fmoc, on the terminal amino acid of the immobilized amino acid is then removed by adding a base to the solution. Side chain functional groups are also protected using any base-stable, acid-labile groups to avoid unwanted reactions. Before the second amino acid is attached to the immobilized amino acid, the amino-group of the second amino acid is blocked, as described above, and the α-carboxyl group of each successive amino acid is activated by creating an N-hydrobenzotriazole (HOBt) ester in situ. The activated α-carboxyl group of the second amino acid and the free amino group of the immobilized amino acid then react, in the presence of a base, to form a new peptide bond. Additional amino acids are then successively added to the terminal amino acid of the immobilized peptide chain, until the desired peptide has been assembled. Once the necessary amino acids have been attached, the peptide chain can be cleaved from the resin, such as for example, by using a mixture of trifluoroacetic acid (TFA) and scavengers (e.g., phenol, thioanisol, water, ethanedithiol (EDT) and triisopropylsilan (TIS)) that are effective to neutralize any cations formed as the protecting groups attached to the side chain functional groups of the assembled peptide chain are removed.

It is well known to those skilled in the art that proteins or peptides that are produced by any of the above methods can be further chemically modified to enhance the protein half-life in the circulation. By way of non-limiting example, polyethylene glycol (PEG) residues can be attached to the penetrating peptides of the invention. Conjugating biomolecules with PEG, a process known as pegylation, is an established method for increasing the circulating half-life of proteins. Polyethylene glycols are nontoxic water-soluble polymers that, because of their large hydrodynamic volume, create a shield around the pegylated molecule, thereby protecting it from renal clearance, enzymatic degradation, as well as recognition by cells of the immune system.

Agent-specific pegylation methods have been used in recent years to produce pegylated molecules (e.g., drugs, proteins, agents, enzymes, etc.) that have biological activity that is the same as, or greater than, that of the “parent” molecule. These agents have distinct in vivo pharmacokinetic and pharmacodynamic properties, as exemplified by the self-regulated clearance of pegfilgrastim, the prolonged absorption half-life of pegylated interferon alpha-2a. Pegylated molecules have dosing schedules that are more convenient and more acceptable to patients, which can have a beneficial effect on the quality of life of patients. (See e.g., Yowell S. L. et aL, Cancer Treat Rev 28 Suppl. A:3-6 (April 2002)).

The invention also includes a method of contacting biological barrier with a penetrating module or peptide in an amount sufficient to enable efficient penetration between the cells. The module or peptide can be provided in vitro, ex vivo, or in vivo. Furthermore, the penetrating peptide according to this invention may be capable of potentializing the biological activity of the coupled substance. Therefore, another purpose of this invention is a method of using penetrating peptides to increase the biological activity of the effector to which it is coupled.

In addition to the peptide-effector modules or peptides and peptide-molecular vessel-effector modules, the invention also provides a pharmaceutically acceptable base or acid addition salt, hydrate, ester, solvate, prodrug, metabolite, stereoisomer, or mixture thereof The invention also includes pharmaceutical formulations comprising a peptide-effector “module” or peptide-molecular vessel-effector module in association with a pharmaceutically acceptable carrier, diluent, protease inhibitor, surface active agent, or excipient.

Salts encompassed within the term “pharmaceutically acceptable salts” refer to non-toxic salts of the compounds of this invention which are generally prepared by reacting the free base with a suitable organic or inorganic acid to produce “pharmaceutically-acceptable acid addition salts” of the compounds described herein. These compounds retain the biological effectiveness and properties of the free bases. Representative of such salts are the water-soluble and water-insoluble salts, such as the acetate, amsonate (4,4-diaminostilbene-2,2′-disulfonate), benzenesulfonate, benzonate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methylene-bis-2-hydroxy-3-naphthoate, embonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

According to the methods of the invention, a human patient can be treated with a pharmacologically effective amount of a peptide or module. The term “pharmacologically effective amount” means that amount of a drug or pharmaceutical agent (the effector) that will elicit the biological or medical response of a tissue, system, animal or human that is being sought by a researcher or clinician.

The invention also includes pharmaceutical compositions suitable for introducing an effector of interest across a biological barrier. The compositions are preferably suitable for internal use and include an effective amount of a pharmacologically active compound of the invention, alone or in combination, with one or more pharmaceutically acceptable carriers. The compounds are especially useful in that they have very low, if any, toxicity.

Preferred pharmaceutical compositions are tablets and gelatin capsules comprising the active ingredient together with a) diluents, e.g. lactose, dextrose, sucrose, mannitol, sorbitol, cellulose and/or glycine; b) protease inhibitors; c) lubricants, e.g., silica, talcum, stearic acid, its magnesium or calcium salt, poloxamer and/or polyethyleneglycol; for tablets also d) binders, e.g., magnesium aluminum silicate, starch paste, gelatin, tragacanth, methylcellulose, sodium carboxymethylcellulose and/or polyvinylpyrrolidone; if desired e) disintegrants, e.g., starches, agar, alginic acid or its sodium salt, or effervescent mixtures; and/or f) absorbents, colorants, flavors and sweeteners. Injectable compositions are preferably aqueous isotonic solutions or suspensions, and suppositories are advantageously prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional mixing, granulating or coating methods, respectively, and contain about 0.01 to 75%, preferably about 0.1 to 10%, of the active ingredient.

Administration of the active compounds and salts described herein can be via any of the accepted modes of administration for therapeutic agents. These methods include systemic administration such as intravenous, or local administration such as oral, bucal, anal, bronchial, nasal, parenteral, transdermal, subcutaneous, or topical administration modes.

Depending on the intended mode of administration, the compositions may be in solid, semi-solid or liquid dosage form, such as, for example, injectables, tablets, suppositories, pills, time-release capsules, powders, liquids, suspensions, aerosol or the like, preferably in unit dosages. The compositions will include an effective amount of active compound or the pharmaceutically acceptable salt thereof, and in addition, may also include any conventional pharmaceutical excipients and other medicinal or pharmaceutical drugs or agents, carriers, adjuvants, diluents, protease inhibitors, etc., as are customarily used in the pharmaceutical sciences.

For solid compositions, excipients include pharmaceutical grades of mannitol, lactose, starch, magnesium stearate, sodium saccharin, talcum, cellulose, glucose, sucrose, magnesium carbonate, and the like may be used. The active compound defmed above, may be also formulated as suppositories using for example, polyalkylene glycols, for example; propylene glycol, as the carrier.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension.

If desired, the pharmaceutical composition to be administered may also contain minor amounts of non-toxic auxiliary substances such as wetting or emulsifying agents, pH buffering agents, and other substances such as for example, sodium acetate, triethanolamine oleate, etc.

Parental injectable administration is generally used for subcutaneous, intramuscular or intravenous injections and infusions. Injectables can be prepared in conventional forms, either as liquid solutions or suspensions or solid forms suitable for dissolving in liquid prior to injection.

One approach for parenteral administration employs the implantation of a slow-release or sustained-released system, which assures that a constant level of dosage is maintained, according to U.S. Pat. No. 3,710,795, incorporated herein by reference.

Those skilled in the art will recognize that the penetrating modules or peptides of the instant invention can be used as an oral vaccine. Such a vaccine may comprise a penetrating peptide coupled with a desired antigenic sequence, including, but not limited to, the PA antigen of Anthrax. This fusion protein is then orally administered to a subject in need of vaccination.

An “antigen” is a molecule or a portion of a molecule capable of stimulating an immune response, which is additionally capable of inducing an animal or human to produce antibody capable of binding to an epitope of that antigen. An “epitope” is that portion of any molecule capable of being recognized by and bound by a major histocompatability complex (“MHC”) molecule and recognized by a T cell or bound by an antibody. A typical antigen can have one or more than one epitope. The specific recognition indicates that the antigen will react, in a highly selective manner, with its corresponding MHC and T cell, or antibody and not with the multitude of other antibodies which can be evoked by other antigens.

A peptide is “immunologically reactive” with a T cell or antibody when it binds to an MHC and is recognized by a T cell or binds to an antibody due to recognition (or the precise fit) of a specific epitope contained within the peptide. Immunological reactivity can be determined by measuring T cell response in vitro or by antibody binding, more particularly by the kinetics of antibody binding, or by competition in binding using known peptides containing an epitope against which the antibody or T cell response is directed as competitors.

Techniques used to determine whether a peptide is immunologically reactive with a T cell or with an antibody are known in the art. Peptides can be screened for efficacy by in vitro and in vivo assays. Such assays employ immunization of an animal, e.g., a mouse, a rabbit or a primate, with the peptide, and evaluation of the resulting antibody titers.

Also included within the invention are vaccines that can elicit the production of secretory antibodies (IgA) against the corresponding antigen, as such antibodies serve as the first line of defense against a variety of pathogens. Oral vaccination, which has the advantages of being a non-invasive route of administration, is the preferred means of immunization for obtaining secretory antibodies.

The compounds of the present invention can be administered in such oral dosage forms as tablets, capsules (each including timed release and sustained release formulations), pills, powders, granules, elixirs, tinctures, suspensions, syrups and emulsions. Likewise, they may also be administered in intravenous (both bolus and infusion), intraperitoneal, subcutaneous or intramuscular form, and all using forms well known to those of ordinary skill in the pharmaceutical arts.

The dosage regimen utilizing the compounds is selected in accordance with a variety of factors including type, species, age, weight, sex and medical condition of the patient; the severity of the condition to be treated; the route of administration; the renal and hepatic finction of the patient; and the particular compound or salt thereof employed. An ordinarily skilled physician or veterinarian can readily determine and prescribe the effective amount of the drug required to prevent, counter or arrest the progress of the condition.

Oral dosages of the present invention, when used for the indicated effects, may be provided in the form of scored tablets containing 0.005, 0.01, 0.025, 0.05, 0.1, 0.25, 0.5, 1.0, 2.5, 5.0, 10.0, 15.0, 25.0, 50.0, 100.0, 250.0, 500.0 or 1000.0 mg of active ingredient.

Compounds of the present invention may be administered in a single daily dose, or the total daily dosage may be administered in divided doses of two, three or four times daily. Furthermore, preferred compounds for the present invention can be administered in bucal form via topical use of suitable bucal vehicles, bronchial form via suitable aerosols or inhalants, intranasal form via topical use of suitable intranasal vehicles, or via trnsdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels, wherein the concentration of active ingredient would range from 0.1% to 15%, w/w or w/v.

The compounds herein described in detail can form the active ingredient, and are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders, lubricants, protease inhibitors, disintegrating agents and coloring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, poloxamer, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methylcellulose, agar, bentonite, xanthan gum and the like.

The compounds of the present invention can also be administered in the form of liposome delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to a form lipid layer encapsulating the drug, as described in U.S. Pat. No. 5,262,564.

The compounds of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Any of the above pharmaceutical compositions may contain 0.01-99%, preferably 0.1-10% of the active compounds as active ingredients.

The following EXAMPLES are presented in order to more fully illustrate the preferred embodiments of the invention. These EXAMPLES should in no way be construed as limiting the scope of the invention, as defined by the appended claims.

### EXAMPLES

### Example 1

### Demonstration of the Efficacy of the Penetrating Module to Enable the Translocation of a Peptide Through an Epithelial Barrier

The tested peptide, representing a penetrating peptide and a radio-labeled probe in a contiguous construct of SEQ ID NO 3 and SEQ ID NO 17, hereby named IBW 002 (Novetide, lot 40139-45, sequence: Ac-N-Y-Y-D-I-T-L-A-L-A-G-I-C-Q-S-A-R-L-V-Q-Q-L-A-G-G-G-K-G-G-K-NH₂ (SEQ ID NO:22), was ¹²⁵I-labeled using the Bolton Hunter Reagent (Biochem. J. 133:529-39 (1973)). The labeled peptide was loaded on a Sephadex G10 column, washed with phosphate buffer and eluted with 3M Tiocyanate in DDW+50% n-butanol. The peak, containing 6 fractions, was kept and used for in vivo experiments.

The test sample was prepared as shown in Table 2

TABLE 2

| | |
|---|---|
| ¹²⁵I-IBW-002 sample | 26 ul |
| PE 6,200 | 20 ul |
| Aprotinin | 100 ul |
| PB | 849 ul |
| NAC (stock 9.8 mg/ml) | 5 ul |

In vivo Experimental Procedure:

Four male BALB/c mice, 9-10 weeks old, were deprived of food, 18 hours prior to the experiment.

The mice were anesthetized by i.p. injection of 0.05 ml of a mixture of 0.15 ml xylazine+0.85 ml of ketamin. A 2 cm long incision was made along the center of the abdomen, through the skin and abdominal wall. An intestine loop was gently pulled out through the incision and placed on wet gauze beside the animal. The loop remained intact through the entire procedure and was kept wet during the whole time. The tested compound was injected into the loop, 0.2 ml/ mouse, containing around 200,000 cpm, using a 26G needle. Fifteen minutes post injection the tip of the tail was cut and a 50 μl blood sample was drawn into a glass capillary. This was repeated 30 and 60 minutes post injection. The animals were subsequently sacrificed. The following organs were removed: epididymal fat, kidneys, liver, lungs and brain. Radioactivity in each organ and in blood samples was counted. A 5 μl sample of the injected solution was counted to determine the total injected cpm per mouse.

Results:

The total radioactive labeling injected per mouse was 224,160 cpm. The cpm recovered from each tissue is summarized in Table 3. Total cpm in the blood was calculated based on the 50 μl blood sample drawn out at different time points post injection, in the assumption that total blood volume of each mouse is 2 ml.

TABLE 3

| | mouse #1 | mouse #2 | mouse #3 | mouse #4 | mean | ±SEM |
|---|---|---|---|---|---|---|
| blood 15′ | 37,080 | 68,960 | 62,280 | 80,280 | 62,150 | 9,145 |
| blood 30′ | 36,120 | 60,680 | 52,320 | 70,840 | 54,990 | 7,342 |
| blood 60′ | 29,720 | 47,800 | 44,000 | 59,720 | 45,310 | 6,182 |
| Epididymal | 547 | 869 | 973 | 1,174 | 891 | 131 |
| fat | | | | | | |
| kidney | 4,068 | 6,614 | 6,400 | 7,650 | 6,183 | 756 |
| liver | 6,102 | 9,709 | 8,718 | 11,674 | 9,051 | 1,159 |
| lungs | 865 | 1,343 | 1,349 | 1,806 | 1,341 | 192 |
| brain | 138 | 190 | 202 | 301 | 208 | 34 |
| recovered | 41,440 | 66,525 | 61,642 | 82,325 | 62,983 | 8,429 |
| cpm/mouse | 18.49 | 29.68 | 27.50 | 36.73 | 28.10 | 3.76 |
| recovered/ | | | | | | |
| injected cpm (%) | | | | | | |

The percent of ¹²⁵I-IBW-002 in the blood relative to the total injected ¹²⁵I-IBW-002, at the 3 different time points, is presented in Table 4.

As can be seen in Table 4, the relative amount of ¹²⁵I-IBW-002 in the blood peaked already at 15 min. post-intestinal administration and decreased with time. This probably indicates peptide distribution from the blood to various organs.

TABLE 4

| | mouse | mouse | mouse | mouse | | |
|---|---|---|---|---|---|---|
| | #1 | #2 | #3 | #4 | mean | ±SEM |
| blood/injected | 16.54 | 30.76 | 27.78 | 35.81 | 27.73 | 4.08 |
| cpm (%), 15′ | | | | | | |
| blood/injected | 16.11 | 27.07 | 23.34 | 31.60 | 24.53 | 3.28 |
| cpm (%), 30′ | | | | | | |
| blood/injected | 13.26 | 21.32 | 19.63 | 26.64 | 20.21 | 2.76 |
| cpm (%), 60′ | | | | | | |

In similar experiments, a control ¹²⁵I-labeled peptide (SEQ ID NO:17), lacking the penetrating peptide-sequence, was injected into a mouse intestinal loop. The average amount of the control peptide recovered from the blood and various organs was only about ⅕ of that obtained with the full conjugate of the IBW-002 peptide, although this control peptide is about ¼ in its size as compared with IBW-002.

### Example 2

### Development of Optimal Formulation.

To reach an improved formulation, a number of compounds were tested in experiments similar to that described in Example 1, with the following change: urine was collected and the measured radioactivity was added to that of the recovered tissues.

a. Non Ionic Detergents (Preferably Poloxamers)

TABLE 5
Initial formulation with Pluronic PE 6,200

| | |
|---|---|
| Peptide sample (radiolabeled) | 10 ul |
| PE 6,200 | 20 ul |
| Aprotinin | 100 ul |
| PB | 5 ul |
| NAC (9.8 mg/ml) | 5 ul |

The total radioactive labeling injected per mouse was 125,320 cpm. The cpm recovered from each tissue is summarized in Table 6. Total cpm in the blood was calculated based on the 50 μl blood sample drawn out at different time points post injection, in the assumption that total blood volume of each mouse is 2 ml.

TABLE 6

| | mouse #1 | mouse #2 | mean | ±SEM |
|---|---|---|---|---|
| blood 15′ | 19,920 | 19,560 | 19,740 | 180 |
| blood 30′ | 18,800 | 15,880 | 17,340 | 1,460 |
| blood 60′ | 14,560 | 10,960 | 12,760 | 1,800 |
| epididimal | 321 | 288 | 305 | 17 |
| fat | | | | |
| kidney | 2,564 | 3,799 | 3,182 | 618 |
| liver | 2,994 | 2,786 | 2,890 | 104 |
| lungs | 476 | 377 | 427 | 50 |
| brain | 95 | 110 | 103 | 8 |
| urine | 14,749 | 21,747 | 18,248 | 3,499 |
| recovered | 35,759 | 40,067 | 37,913 | 2,154 |
| cpm/mouse | | | | |
| recovered | 28.53 | 31.97 | 30.25 | 1.72 |
| cpm/ | | | | |
| injected cpm | | | | |
| (%) | | | | |

The percent of ¹²⁵I-IBW-002 in the blood relative to the total injected ¹²⁵I-IBW-002, at the 3 different time points, is presented in Table 7

TABLE 7

| | mouse #1 | mouse #2 | mean | ±SEM |
|---|---|---|---|---|
| blood/injected cpm (%), 15′ | 15.90 | 15.61 | 15.75 | 0.14 |
| blood/injected cpm (%), 30′ | 15.00 | 12.67 | 13.84 | 1.17 |
| blood/injected cpm (%), 60′ | 11.62 | 8.75 | 10.18 | 1.44 |

Calculated total recovery was 30.25%.

b. Bile Salts (Preferably Taurodeoxycholic Acid)

TABLE 8

| | | |
|---|---|---|
| Peptide sample (radiolabeled) | 10 | ul |
| PE 6,200 | 20 | ul |
| Aprotinin | 100 | ul |
| PB | 802.5 | ul |
| NAC 9.8 mg/ml | 5 | ul |
| Taurodeoxycholic acid (8% in DDW) | 62.5 | ul |

The total radioactive labeling injected per mouse was 195,900 cpm. The cpm recovered from each tissue is summarized in Table 9. Total cpm in the blood was calculated based on the 50 μl blood sample drawn out at different time points post injection, in the assumption that total blood volume of each mouse is 2 ml.

TABLE 9

| | mouse #1 | mouse #2 | mouse #3 | mouse #4 | mean | ±SEM |
|---|---|---|---|---|---|---|
| blood 15′ | 40,600 | 39,440 | 51,920 | 36,200 | 42,040 | 3,422 |
| blood 30′ | 37,440 | 31,720 | 48,440 | 34,720 | 38,080 | 3,646 |
| blood 60′ | 33,880 | 23,080 | 40,840 | 30,200 | 32,000 | 3,702 |
| epididimal fat | 1,081 | 805 | 903 | 846 | 909 | 61 |
| kidney | 9,513 | 7,878 | 10,661 | 8,929 | 9,245 | 581 |
| liver | 8,194 | 5,154 | 9,615 | 8,708 | 7,918 | 967 |
| lungs | 1,048 | 636 | 1,221 | 1,303 | 1,052 | 149 |
| brain | 206 | 176 | 284 | 173 | 210 | 26 |
| urine | 39,992 | 30,651 | 25,445 | 40,361 | 34,112 | 3,660 |
| recovered | 93,914 | 68,380 | 89,969 | 90,520 | 85,446 | 5,782 |
| cpm/mouse | 47.94 | 34.91 | 45.42 | 46.21 | 43.62 | 2.95 |
| recovered cpm/ | | | | | | |
| injected cpm (%) | | | | | | |

The percent of ¹²⁵I-IBW-002 in the blood relative to the total injected ¹²⁵I-IBW-002, at the 3 different time points, is presented in Table 10.

TABLE 10

| | mouse | mouse | mouse | mouse | | |
|---|---|---|---|---|---|---|
| | #1 | #2 | #3 | #4 | mean | ±SEM |
| blood/injected | 20.72 | 20.13 | 26.50 | 18.48 | 21.46 | 1.75 |
| cpm (%), 15′ | | | | | | |
| blood/injected | 19.11 | 16.19 | 24.73 | 17.72 | 19.44 | 1.86 |
| cpm (%), 30′ | | | | | | |
| blood/injected | 17.29 | 11.78 | 20.85 | 15.42 | 16.33 | 1.89 |
| cpm (%), 60′ | | | | | | |

Calculated total recovery was 43.62%.

c. Pluronic F-68 as a Preferred Poloxamer

TABLE 11

| | | |
|---|---|---|
| Peptide sample (radiolabeled) | 18 | ul |
| Pluronic F-68 (2.72% in PB) | 735 | ul |
| Aprotinin | 100 | ul |
| PB | 80 | ul |
| NAC (9.8 mg/ml) | 5 | ul |
| Taurodeoxycholic acid (8% in DDW) | 62.5 | ul |

The total radioactive labeling injected per mouse was 143,000 cpm. The cpm recovered from each tissue is summarized in Table 12. Total cpm in the blood was calculated based on the 50 μl blood sample drawn out at different time points post injection, in the assumption that total blood volume of each mouse is 2 ml.

TABLE 12

| | mouse #1 | mouse #2 | mouse #3 | mouse #4 | mean | ±SEM |
|---|---|---|---|---|---|---|
| blood 5′ | 29,400 | 29,120 | 34,240 | 32,920 | 31,420 | 1,277 |
| blood 10′ | 26,680 | 32,320 | 28,200 | 27,760 | 28,740 | 1,235 |
| blood 20′ | 20,280 | 30,960 | 27,240 | 21,800 | 25,070 | 2,467 |
| blood 60′ | 11,880 | 22,840 | 16,280 | 14,440 | 16,360 | 2,341 |
| epididimal fat | 375 | 591 | 462 | 465 | 473 | 44 |
| kidney | 2,397 | 4,407 | 5,018 | 5,772 | 4,399 | 723 |
| liver | 3,516 | 6,546 | 6,561 | 4,807 | 5,358 | 739 |
| lungs | 393 | 643 | 582 | 533 | 538 | 53 |
| brain | 76 | 106 | 94 | 74 | 88 | 8 |
| urine | 59,442 | 32,158 | 35,831 | 53,271 | 45,176 | 6,620 |
| recovered | 78,079 | 67,291 | 64,828 | 79,362 | 72,390 | 3,699 |
| cpm/mouse | 54.60 | 47.06 | 45.33 | 55.50 | 50.62 | 2.59 |
| recovered cpm/ | | | | | | |
| injected cpm (%) | | | | | | |

The percent of ¹²⁵I-IBW-002 in the blood relative to the total injected ¹²⁵I-IBW-002, at the four different time points, is presented in Table 13.

TABLE 13

| | mouse | mouse | mouse | mouse | | |
|---|---|---|---|---|---|---|
| | #1 | #2 | #3 | #4 | mean | ±SEM |
| blood/injected | 20.56 | 20.36 | 23.94 | 23.02 | 21.97 | 0.89 |
| cpm (%), 5′ | | | | | | |
| blood/injected | 18.66 | 22.60 | 19.72 | 19.41 | 20.10 | 0.86 |
| cpm (%), 10′ | | | | | | |
| blood/injected | 14.18 | 21.65 | 19.05 | 15.24 | 17.53 | 1.73 |
| cpm (%), 20′ | | | | | | |
| blood/injected | 8.31 | 15.97 | 11.38 | 10.10 | 11.44 | 1.64 |
| cpm (%), 60′ | | | | | | |

Calculated total recovery was 50.62%.

TABLE 14
Summary of formulation results regarding the added detergents

| Formulation | Recovery (%) |
|---|---|
| 2% PE 6,200 | 30.25 |
| 2% PE 6,200 + 0.5% Taurodeoxycholic acid | 43.62 |
| 2% Pluronic F-68 + 0.5% Taurodeoxycholic acid | 50.62 |

### Example 3

### Relative Potency of Penetrating Peptides as Tested in Mice.

TABLE 15

| | | | |
|---|---|---|---|
| | SEQ | | Relative |
| Peptide's | ID | | potency |
| name | NO. | Sequence | (%) |

| | | | |
|---|---|---|---|
| IBW-002 | 22 | AcNYYDITLALAGICQSARLVQQLAGGGK | 100 |
| | | GGKNH₂ | |
| IBW-002V2 | 36 | AcMYYDITLALAGICQSARLVQQLAGGGK | 80 |
| | | GGKNH₂ | |
| IBW-002V3 | 37 | AcMYDITLALAGICQSARIVQQLAGGKGG | 77 |
| | | KNH₂ | |
| IBW-006 | 33 | AcNYHDIVLALAGVCQSARLVHQLAGGKG | 77 |
| | | GKNH₂ | |
| IBW-002V1 | 35 | AcMNYYDITLALAGICQSARLVQQLAGGG | 73 |
| | | KGGKNH₂ | |
| IBW-007 | 34 | AcNYFLVNLAFAEASMAAFNTVVNFGGKG | 66 |
| | | GKNH₂ | |
| IBW-004 | 31 | AcNYFIVNLALADLCMAAFNAAFNFGGGK | 63 |
| | | GGKNH₂ | |
| IBW-005 | 32 | AcMRNLTRTSLLLAGLCTAAQMVFVGGGK | 51 |
| | | GGKNh₂ | |
| IBW-003 | 30 | AcNLPPIVLAVIGICAAVFLLQQYVGGGK | 37 |
| | | GGKNH₂ | |

### Example 4

### Conditionally-activated Effectors: Inhibition of Excessive Blood Coagulation

SEQ ID NO:3 (or any other sequence from SEQ ID NO:1-15, 24-29) is coupled to a coagulation inhibitory protein (e.g., hirudin, hirulog, Protein C, Protein C activator or Protein S) or a thrombolysis activating protein (ie., tPA) through the sequence IEGR (SEQ ID NO: 16), a cleavage site recognized by factor Xa.

At sites of excessive blood coagulation, the linker sequence will be cleaved by the abundant factor Xa, thereby releasing the coupled coagulation inhibitory protein. As a result, the coagulation process will be attenuated. Inhibition of excessive or pathological coagulation can be assessed locally by reduction in thrombus size, morphology, and propagation along the vasculature.

Consequently, a reduction of ischemic damage can be demonstrated via histological and functional extent of an infarct. Systemic effects can be assessed by lengthened Prothromin Time (PT) and Partial Thromboplastin Time (PTT), and reduced levels of fibrinogen degradation products and D-dimers (FDPs).

### Example 5

### Conditionally-activated Effectors: Inhibition of Pathologic Inflammatory Processes

SEQ ID NO:3 (or any other sequence from SEQ ID NO:1-15, 24-29) is coupled to a complement inhibitory protein (e.g., a Clq inhibitor) through a cleavage site that is recognized by complement cascade proteins (e.g., C3a).

At instances of pathological complement activation, such as excessive inflammatory processes, the complement inhibitor will be released. Inhibition of the complement cascade can be assessed directly by a reduction in CH50, or individual C3 and C4 activity. Overall modulation of inflammation may be determined by a reduction in levels of acute phase proteins, e.g., C-reactive protein (CRP), or a reduction in elevated sedimentation rate (ESR).

### Example 6

### Utilization of the Penetrating Module for Oral Vaccination

SEQ ID NO:3 (or any other sequence from SEQ ID NO:1-15, 24-29) is coupled to a desired antigenic sequence. For example, a fusion protein composed of the penetrating module coupled to the PA antigen of Anthrax. Such a fusion protein can be administered to a subject in need of vaccination.

This method allows simple and rapid vaccination of large populations in need thereof. Another advantage of this method is the production of high titers of IgA antibodies and the subsequent presence of IgA antibodies in the epithelial mucosa, which are the sites of exposure to antigens.

Efficacy of vaccination can be demonstrated by the measurement of specific antibody titers, especially for IgA, as well as the measurement of immunological response to stimulation, such as for example, via a cutaneous hyerpsensitivity reaction in response to subcutaneous administration of antigen.

### Example 7

### Utilization of the Penetrating Module for Bulk Translocation

By covalently attaching stearyl residues to the free amino groups of the two lysines at the C-terminus of SEQ ID NO:22, a hydrophobized penetrating peptide is produced. This hydrophobized penetrating peptide is then incorporated into the interface of taxol-containing microparticles composed of triglyceride medium.

This method allows for the translocation of highly hydrophobic drugs, such as taxol, from the intestine into the bloodstream. Because such drugs cannot be otherwise absorbed from the intestine, they are typically administered to patients via invasive means.

The effects of taxol-containing microparticles can be demonstrated by a reduction in tumor size, metastases or other tumor-related markers.

### Example 8

### Recombinant Human Insulin (rh-Insulin) Delivery Across Mucosal Epithelia via a Fusion Construct

SEQ ID NO:3 (or any other sequence from SEQ ID NO:1-15, 24-29) is coupled to rh-Insulin in one of two ways:

1. through a cleavage site:

(SEQ ID NO:18)

NYYDITLALAGICQSARLVQQLA-GG-IEGR- rh-Insulin

2. without a cleavage site:

(SEQ ID NO:19)

NYYDITLALAGICQSARLVQQLA-GG- rh-Insulin.

The resulting compound is dissolved in a 2% pluronic PE 6200 aqueous solution. Two hundred μL of this solution, (or vehicle alone), is injected into an intestinal loop of a mouse and blood glucose levels are subsequently measured.

Blood glucose levels decrease in relation to the amount of insulin absorbed from the intestine into the bloodstream (i.e., in an amount that correlates to the amount of insulin absorbed). Thus, this drug delivery system can replace the need for insulin injections, thereby providing an efficient, safe and convenient route of administration for diabetes patients.

### Example 9

### Recombinant Human Insulin (rh-Insulin) Delivery Across Mucosal Epithelia via a Molecular Vessel Fusion Construct

SEQ ID NO:3 (or any other sequence from SEQ ID NO:1-15, 24-29) is coupled to a ligand binding domain of the insulin receptor (or minireceptor) which, in turn, is bound to rh-Insulin as follows:

NYYDITLALAGICQSARLVQQLA-GG- linearized insulin receptor (SEQ ID NO:23) (see Kristensen, et al., J. Biol. Chem., 274(52):37351-56 (1999)) plus an equimolar amount of rh-insulin.

The resulting complex is dissolved in a 2% pluronic PE 6200 aqueous solution. Two hundred μL of the above dissolved complex, (or vehicle alone), is injected into an intestinal loop of a mouse and blood glucose levels are subsequently measured.

Blood glucose levels decrease in relation to the amount of insulin absorbed from the intestine into the bloodstream (ie., in an amount that correlates to the amount of insulin absorbed). Thus, this drug delivery system can replace the need for insulin injections, thereby providing an efficient, safe and convenient route of administration for diabetes patients.

### Example 10

### Low Molecular Weight Heparin Delivery Across Mucosal Epithelia

SEQ ID NO:3 (or any other sequence from SEQ ID NO:1-15, 24-29) is coupled to heparin for example, through the free amino group of an extra lysine added at the C-terminus, in one of two ways:

1. through a cleavage site:

(SEQ ID NO:20)

NYYDITLALAGICQSARLVQQLA-GG-IEGR-K- heparin

2. without a cleavage site:

(SEQ ID NO:21)

NYYDITLALAGICQSARLVQQLA-GG-K- heparin.

The resulting compound is dissolved in a 2% pluronic PE 6200 aqueous solution. Two hundred μL of this solution, (or vehicle alone), is injected into an intestinal loop of a mouse. Partial Thrombin Time (PTT) values are subsequently measured.

Partial Thrombin Time (PTT) values decrease in relation to the amount of heparin absorbed from the intestine loop into the bloodstream (ie., in an amount that correlates to the amount of insulin absorbed). Therefore, this drug delivery system will replace the use of heparin injections.

### OTHER EMBODIMENTS

From the foregoing detailed description of the specific embodiments of the invention, it should be apparent that unique methods of translocation across epithelial and endothelial barriers have been described. Although particular embodiments have been disclosed herein in detail, this has been done by way of example for purposes of illustration only, and is not intended to be limiting with respect to the scope of the appended claims that follow. In particular, it is contemplated by the inventor that various substitutions, alterations, and modifications may be made to the invention without departing from the spirit and scope of the invention as defined by the claims. For instance, the choice of the particular type of tissue, or the particular effector to be translocated is believed to be a matter of routine for a person of ordinary skill in the art with knowledge of the embodiments described herein.

1. A penetrating peptide comprising at least one amino acid sequence selected from the group consisting of:
a) (BX)₄Z(BX)₂ZXB;
b) ZBXB₂XBXB₂XBX₃BXB₂X₂B₂;
c) ZBZX₂B₄XB₃ZXB₄Z₂B₂;
d) ZB₉XBX₂B₂ZBXZBX₂;
e) BZB₈XB₉X₂ZXB;
f) B₂ZXZB₅XB₂XB₂X₂BZXB₂;
g) XB₉XBXB₆X₃B;
h) X₂B₃XB₄ZBXB₄XBₙXB;
i) XB₂XZBXZB₂ZXBX₃BZXBX₃B;
j) BZXBXZX₂B₄XBX₂B₂XB₄X₂;
k) BZXBXZX₂B₄XBX₂B₂XB₄;
l) B₂XZ₂XB₄XBX₂B₅X₂B₂;
m) B_{q}XₜZBₘX_{q}B₄XBXₙBₘZB₂X₂B₂;
n) B₂ZX₃ZBₘX_{q}B₄XBXₙBₘZB₂X₂B₂;
o) X₃ZB₆XBX₃BZB₂X₂B₂; and
p) at least 12 contiguous amino acids of any of peptides a) through o)
wherein
q is 0 or 1;
m is 1 or 2;
n is 2 or 3;
t is 1 or 2 or 3; and
X is any amino acid;
B is a hydrophobic amino acid; and
Z is a charged amino acid;
wherein said penetrating peptide is capable of translocating across a biological barrier.

2. The penetrating peptide of claim 1, wherein the amino acid sequence is selected from the group consisting of:
a) SEQ ID NOS: 1, 2, 3, 4, 5, 6, 7, 8, 9, 10, 11, 12, 13, 14, 15, 24, 25, 26, 27, 28 and 29;
b) a variant of an amino acid sequence selected from the group consisting of SEQ ID NOS: 1, 2, 3, 4, 5, 6, 7, 8, 9, 10, 11, 12, 13, 14, 15, 24, 25, 26, 27, 28 and 29, wherein one or more amino acid residues in said variant differs from the amino acid sequence of said penetrating peptide, provided that said variant differs in no more than 15% of amino acid residues from said amino acid sequence;
c) a fragment of an amino acid sequence selected from the group consisting of SEQ ID NOS: 1, 2, 3, 4, 5, 6, 7, 8, 9, 10, 11, 12, 13, 14, 15, 24, 25, 26, 27, 28 and 29; and
d) a peptide comprising at least 12 contiguous amino acids of any of the peptides selected from the group consisting of SEQ ID NOS: 1, 2, 3, 4, 5, 6, 7, 8, 9, 10, 11, 12, 13, 14, 15, 24, 25, 26, 27, 28 and 29.

3. The penetrating peptide of claim 2, wherein the fragment is at least 10 amino acids in length.

4. The penetrating peptide of claim 2, wherein the amino acid sequence of said variant comprises a conservative amino acid substitution.

5. The penetrating peptide of claim 2, wherein the amino acid sequence of said variant comprises a non-conservative amino acid substitution.

6. The penetrating peptide of claim 1, wherein the amino acid sequence is SEQ ID NO: 3 or at least 12 contiguous amino acids thereof.

7. The penetrating peptide of claim 1, wherein the amino acid sequence is SEQ ID NO: 8 or at least 12 contiguous amino acids thereof.

8. The penetrating peptide of claim 1, wherein the amino acid sequence is SEQ ID NO: 9 or at least 12 contiguous amino acids thereof.

9. The penetrating peptide of claim 1, wherein the amino acid sequence is SEQ ID NO: 12 or at least 12 contiguous amino acids thereof.

10. The penetrating peptide of claim 1, wherein the amino acid sequence is SEQ ID NO: 24 or at least 12 contiguous amino acids thereof.

11. The penetrating peptide of claim 1, wherein the peptide is less than 30 amino acids long.

12. The penetrating peptide of claim 1, wherein the peptide is less than 25 amino acids long.

13. The penetrating peptide of claim 1, wherein the peptide is less than 20 amino acids long.

14. A penetrating peptide comprising at least one amino acid sequence selected from the group consisting of:
a) (BX)₄Z(BX)₂ZXB;
b) ZBXB₂XBXB₂XBX₃BXB₂X₂B₂;
c) ZBZX₂B₄XB₃ZXB₄Z₂B₂;
d) ZB₉XBX₂B₂XBXZBX₂;
e) BZB₈XB₉X₂ZXB;
f) B₂ZXZB₅XB₂XB₂X₂BZXB₂;
g) XB₉XBXB6X₃B;
h) X₂B₃XB₄ZBXB₄XBₙXB;
i) XB₂XZBXZB₂ZXBX₃BZXBX₃B;
j) BZXBXZX₂B₄XBX₂B₂XB₄X₂;
k) BZXBXZX₂B₄XBX₂B₂XB₄;
l) B₂XZ₂XB4XBX₂B₅X₂B₂;
m) B_{q}XₜZBₘX_{q}B₄XBXₙBₘZB₂X₂B₂;
n) B₂ZX₃ZBₘX_{q}B₄XBXₙBₘZB₂X₂B₂;
o) X₃ZB₆XBX₃BZB₂X₂B₂; and
p) at least 12 contiguous amino acids of any of peptides a) through o)
wherein
q is 0 or 1;
m is 1 or 2;
n is 2 or 3;
t is 1 or 2 or 3; and
X is any amino acid;
B is a hydrophobic amino acid; and
Z is a charged amino acid;
wherein said penetrating peptide is capable of translocating across a biological barrier and wherein the penetrating peptide is coupled to an effector.

15. The penetrating peptide of claim 14, wherein said effector is a bioactive peptide.

16. The penetrating peptide of claim 15, wherein said bioactive peptide is selected from the group consisting of: insulin, growth hormone, a gonadotropin, a growth factor, erythropoietin, granulocyte/monocyte colony stimulating factor (GM-CSF), αMSH, enkephalin, dalargin, kyotorphin, EPO, bFGF, hirulog, a lutenizing hormone releasing hormone (LHRH) analog, and neurotrophic factors.

17. The penetrating peptide of claim 14, wherein said effector is a pharmaceutically active agent.

18. The penetrating peptide of claim 17, wherein said pharmaceutically active agent is selected from the group consisting of: an anticoagulant, a toxin, an antibiotic, an antipathogenic agent, an antigen, an antibody fragment, an immunomodulator, a vitamin, an enzyme, an antineoplastic agent, and a therapeutic agent.

19. A method of translocating a penetrating peptide across a biological barrier, wherein the penetrating peptide is the penetrating peptide of claim 2.

20. A method of translocating a penetrating peptide across a biological barrier, wherein the penetrating peptide is the penetrating peptide of claim 6.

21. A method of translocating a penetrating peptide across a biological barrier, wherein the penetrating peptide is the penetrating peptide of claim 7.

22. A method of translocating a penetrating peptide across a biological barrier, wherein the penetrating peptide is the penetrating peptide of claim 8.

23. A method of translocating a penetrating peptide across a biological barrier, wherein the penetrating peptide is the penetrating peptide of claim 9.

24. A method of translocating a penetrating peptide across a biological barrier, wherein the penetrating peptide is the penetrating peptide of claim 10.

25. A penetrating peptide comprising at least one penetrating peptide having an amino acid sequence selected from the group consisting of:
a) (BX)₄Z(BX)₂ZXB;
b) ZBXB₂XBXB₂XBX₃BXB₂X₂B₂;
c) ZBZX₂B₄XB₃ZXB₄Z₂B₂;
d) ZB₉XBX₂B₂XBXZBX₂;
e) BZB₈XB₉X₂ZXB;
f) B₂ZXZB₅XB₂XB₂X₂BZXB₂;
g) XB₉XBXB₆X₃B;
h) X₂B₃XB₄ZBXB₄XBₙXB;
i) XB₂XZBXZB₂ZXBX₃BZXBX₃B;
j) BZXBXZX₂B₄XBX₂B₂XB₄X₂;
k) BZXBXZX₂B₄XBX₂B₂XB₄;
l) B₂XZ₂XB₄XBX₂B₅X₂B₂;
m) B_{q}XₜZBₘX_{q}B₄XBXₙBₘZB₂X₂B₂;
n) B₂ZX₃ZBₘX_{q}B₄XBXₙBₘZB₂X₂B₂;
o) X₃ZB₆XBX₃BZB₂X₂B₂; and
p) at least 12 contiguous amino acids of any of peptides a) through o)
wherein
q is 0 or 1;
m is 1 or 2;
n is 2 or 3;
t is 1 or 2 or 3; and
X is any amino acid;
B is a hydrophobic amino acid; and
Z is a charged amino acid;
wherein said penetrating peptide is capable of translocating across a biological barrier and wherein said penetrating peptide is fused to an effector.

26. The penetrating peptide as in any one of claims 1, 14, and 25, wherein translocation across a biological barrier occurs within a tissue selected from the group consisting of: epithelial cells and endothelial cells.

27. The penetrating peptide as in any one of claims 1, 14 and 25, wherein said biological barrier is selected from the group consisting of: tight junctions and the plasma membrane.

28. The penetrating peptide of claim 25, wherein the effector is selected from the group consisting of: a bioactive peptide and a pharmaceutically active agent.

29. The penetrating peptide of claim 28, wherein said bioactive peptide is selected from the group consisting of: insulin, growth hormone, a gonadotropin, a growth factor, erythropoietin, GM-CSF, αMSH, enkephalin, dalargin, kyotorphin, EPO, bFGF, hirulog, an LHRH analog, and neurotrophic factors.

30. The penetrating peptide of claim 28, wherein said pharmaceutically active agent is selected from the group consisting of: an anticoagulant, a toxin, an antibiotic, an antipathogenic agent, an antigen, an antibody fragment, a vitamin, an immunomodulator, an enzyme, an antineoplastic agent, heparin, methotraxate, and a therapeutic agent.

31. A pharmaceutical composition comprising a therapeutically or prophylactically effective amount of the penetrating peptide according to claim 25, and a pharmaceutically acceptable carrier.

32. The pharmaceutical composition of claim 31, wherein the composition further comprises a mixture of at least two substances selected from the group consisting of a non-ionic detergent, an ionic detergent, a protease inhibitor; and a reducing agent.

33. The pharmaceutical composition of claim 32, wherein the non-ionic detergent is a poloxamer.

34. The pharmaceutical composition of claim 33, wherein the poloxamer is pluronic F-68.

35. The pharmaceutical composition of claim 32, wherein the ionic detergent is a bile salt.

36. The pharmaceutical composition of claim 35, wherein the bile salt is Taurodeoxychilate.

37. The pharmaceutical composition of claim 32, wherein the protease inhibitor is selected from the grove consisting of aprotonin and soya bean trypsin inhibitor.

38. The pharmaceutical composition of claim 32, wherein the reducing agent is NAC.

39. A method of producing a penetrating peptide comprising the penetrating peptide of claim 1 and an effector, said method comprising coupling said effector to said penetrating peptide.

40. The method of claim 39, wherein the coupling of said effector is achieved by a covalent bond.

41. The method of claim 40, wherein said covalent bond is a peptide bond.

42. The method of claim 40, wherein the covalent bond is achieved by a homo- or a hetero-finctional bridging reagent.

43. The method of claim 42, wherein the bridging reagent is a succinimidyl-(N-maleimidomethyl) cyclohexane-1-carboxylate (SMCC)-type reagent.

44. The method of claim 40, wherein the covalent bond is achieved by a peptide linker.

45. The method of claim 44, wherein the peptide linker has the sequence of SEQ ID NO: 16 or SEQ ID NO:17.

46. The method of claim 44, wherein the peptide linker can be cleaved by an enzyme.

47. The method of claim 46, wherein the peptide linker is designed to be cleaved by an enzyme conditionally activated under a certain physiological state and wherein the released effector favorably influences that physiological state.

48. The method of claim 39, wherein the coupling of said effector is achieved by a non-covalent bond.

49. The method of claim 48, wherein the non-covalent bond is achieved by an attachment of a hydrophobic moiety to the penetrating peptide, wherein the hydrophobic moiety enables penetrating module to be incorporated at the interface of a hydrophobic vesicle in which the effector is contained.

50. The method of claim 48, wherein the non-covalent bond is the result of a biotin-avidin or biotin-streptavidin interaction.

51. A method of translocating an effector across a biological barrier, said method comprising:
a) coupling said effector to the penetrating peptide of claim 1 to form a penetrating module; and
b) introducing said penetrating module to the biological barrier.

52. A method of oral vaccination, wherein the method comprises administering to subject the penetrating peptide of claim 1 coupled with a desirable antigen.

53. A kit comprising, in one or more containers, a therapeutically or prophylactically effective amount of the pharmaceutical composition of claim 31.

54. A method of treating or preventing a disease or pathological condition, said method comprising administering to a subject in which such treatment or prevention is desired, the pharmaceutical composition of claim 31, in an amount sufficient to treat or prevent said disease or said pathological condition in said subject.

55. The method of claim 54, wherein said disease or said pathological condition is selected from a group consisting of endocrine disorders, diabetes, infertility, hormone deficiencies, osteoporosis, neurodegenerative disorders, Alzheimer's disease, Parkinson's disease, Huntington's disease, cardiovascular disorders, atherosclerosis, hypercoagulable states, hypocoagulable states, coronary disease, cerebrovascular events, metabolic disorders, obesity, vitamin deficiencies, haematological disorders, and neoplastic disease.

56. A method for producing the penetrating peptide of claim 25 comprising
a) transfecting a production cell with a vector comprising a nucleic acid molecule of a fusion protein encoding said penetrating peptide and an effector operably linked to an expression control sequence;
b) culturing said production cell under conditions that permit production of a fusion protein consisting of the penetrating peptide and an effector peptide; and
c) isolating said fusion protein.

57. A peptide comprising an amino acid sequence, wherein said peptide is derived from a pathogenic bacteria, and said peptide is characterized by the ability to penetrate biological barriers in vivo.

58. The peptide of claim 57, wherein the peptide is derived from an integral membrane protein.

59. The peptide of claim 57, wherein the peptide is derived from a bacterial toxin.

60. A peptide comprising an amino acid sequence, wherein said peptide is derived from a nonpathogenic bacteria, and said peptide is characterized by the ability to penetrate biological barriers in vivo.

61. The peptide of claim 60, wherein said peptide is derived from an integral membrane protein.

62. The peptide of claim 60, wherein the peptide is derived from an extracellular protein.

63. A peptide comprising an amino acid sequence, wherein said peptide is derived from a human neurokinin receptor, and said peptide is characterized by the ability to penetrate biological barriers in vivo.

64. A penetrating module comprising at least one penetrating peptide having an amino acid sequence selected from the group consisting of:
a) (BX)₄Z(BX)₂ZXB;
b) ZBXB₂XBXB₂XBX₃BXB₂X₂B₂;
c) ZBZX₂B₄XB₃ZXB₄Z₂B₂;
d) ZB₉XBX₂B₂XBXZBX₂;
e) BZB₈XB₉X₂ZXB;
f) B₂ZXZB₅XB₂XB₂X₂BZXB₂;
g) XB₉XBXB₆X₃B;
h) X₂B₃XB₄ZBXB₄XBₙXB;
i) XB₂XZBXZB₂ZXBX₃BZXBX₃B;
j) BZXBXZX₂B₄XBX₂B₂XB₄X₂;
k) BZXBXZX₂B₄XBX₂B₂XB₄;
l) B₂XZ₂XB₄XBX₂B₅X₂B₂;
m) B_{q}XₜZBₘX_{q}B₄XBXₙBₘZB₂X₂B₂;
n) B₂ZX₃ZBₘX_{q}B₄XBXₙBₘZB₂X₂B₂;
o) X₃ZB₆XBX₃BZB₂X₂B₂; and
p) at least 12 contiguous amino acids of any of peptides a) through o)
wherein
q is 0 or 1;
m is 1 or 2;
n is 2 or 3;
t is 1 or 2 or 3; and
X is any amino acid;
B is a hydrophobic amino acid; and
Z is a charged amino acid;
wherein said penetrating module is capable of translocating across a biological barrier and wherein said penetrating peptide is fused to molecular vessel, wherein said molecular vessel encloses an effector.

65. The penetrating module of claim 64, wherein the effector is selected from the group consisting of: a bioactive peptide and a pharmaceutically active agent.

66. The penetrating module of claim 65, wherein said bioactive peptide is selected from the group consisting of: insulin, growth hormone, a gonadotropin, a growth factor, erythropoietin, GM-CSF, αMSH, enkephalin, dalargin, kyotorphin, EPO, bFGF, hirulog, an LHRH analog, and neurotrophic factors.

67. The penetrating module of claim 65, wherein said pharmaceutically active agent is selected from the group consisting of: an anticoagulant, a toxin, an antibiotic, an antipathogenic agent, an antigen, an antibody fragment, a vitamin, an immunomodulator, an enzyme, an antineoplastic agent, heparin, methotraxate, and a therapeutic agent.

68. The penetrating module of claim 64, wherein the molecular vessel is selected from the group consisting of a soluble receptor, a minireceptor, and a binding protein.

69. The penetrating module of claim 68, wherein the soluble receptor is a soluble insulin receptor.

70. The penetrating module of claim 69, wherein the effector is insulin.

71. The penetrating module of claim 68, wherein the minireceptor is the ligand-binding domain of the insulin receptor.

72. The penetrating module of claim 71, wherein the effector is insulin.

73. The penetrating module of claim 68, wherein the binding protein is Intrinsic factor.

74. The penetrating module of claim 73, wherein the effector is vitamin B12.

75. A pharmaceutical composition comprising a therapeutically or prophylactically effective amount of the penetrating module according to claim 64, and a pharmaceutically acceptable carrier.

76. A method for producing the penetrating peptide of claim 25 comprising using solid-phase synthesis of the peptide.

77. The penetrating peptide of claim 15, further comprising a chemical modification.

78. The penetrating peptide of claim 77, wherein said bioactive peptide is selected from the group consisting of: insulin, growth hormone, a gonadotropin, a growth factor, erythropoietin, granulocytelmonocyte colony stimulating factor (GM-CSF), αMSH, enkephalin, dalargin, kyotorphin, EPO, bFGF, hirulog, a lutenizing hormone releasing hormone (LHRH) analog, and neurotrophic factors.

79. The method of claim 56, wherein the fusion protein is further chemically modified.

80. The method of claim 79, wherein the chemical modification comprises the attachment of one or more polyethylene glycol residues to the fusion protein.


SEQUENCE LISTING (72 sequences)

SEQ ID NO: 1 - protein, 23 aa, Haemophilus influenzae
NYHDIVLALAGVCQSAKLVHQLA

SEQ ID NO: 2 - protein, 23 aa, Pasteurella multocida
NYYDITLALAGVCQAAKLVQQFA

SEQ ID NO: 3 - protein, 23 aa, Escherichia coli
NYYDITLALAGICQSARLVQQLA

SEQ ID NO: 4 - protein, 23 aa, Vibrio cholerae
AIYDRTIAFAGICQAVALVQQVA

SEQ ID NO: 5 - protein, 23 aa, Buchnera aphidicola
KIHLITLSLAGICQSAHLVQQLA

SEQ ID NO: 6 - protein, 23 aa, Pseudomonas aeruginosa
DPRQQLIALGAVFESAALVDKLA

SEQ ID NO: 7 - protein, 23 aa, Xylella fastidiosa
LIDNRVLALAGVVQALQQVRQIA

SEQ ID NO: 8 - protein, 23 aa, Rhizobium loti
NLPPIVLAVIGICAAVFLLQQYV

SEQ ID NO: 9 - protein, 23 aa, Homo sapiens
NYFIVNLALADLCMAAFNAAFNF

SEQ ID NO: 10 - protein, 23 aa, Chlamydia pneumoniae
TAFDFNKMLDGVCTYVKGVQQYL

SEQ ID NO: 11 - protein, 23 aa, Rhizobium loti
RAILIPLALAGLCQVARAGDISS

SEQ ID NO: 12 - protein, 25 aa, Bacillus subtilis
MRNLTKTSLLLAGLCTAAQMVFVTH

SEQ ID NO: 13 - protein, 25 aa, Kingella denitrificans
IELMIVIAIIGILAAIALPAYQEYV

SEQ ID NO: 14 - protein, 25 aa, Eikenella corrodens
IELMIVIAIIGILAAIALPAYQDYV

SEQ ID NO: 15 - protein, 16 aa, Zonula occludens toxin
ASFGFCIGRLCVQDGF

SEQ ID NO: 16 - protein, 4 aa, Artificial sequence
  Synthetic Cleavable linker peptide
IEGR

SEQ ID NO: 17 - protein, 6 aa, Artificial sequence
  Synthetic Cleavable linker peptide
GGKGGK

SEQ ID NO: 18 - protein, 29 aa, Artificial sequence
  Synthetic penetrating peptide
NYYDITLALAGICQSARLVQQLAGGIEGR

SEQ ID NO: 19 - protein, 25 aa, Artificial sequence
  Synthetic penetrating peptide
NYYDITLALAGICQSARLVQQLAGG

SEQ ID NO: 20 - protein, 30 aa, Artificial sequence
  Synthetic penetrating peptide
NYYDITLALAGICQSARLVQQLAGGIEGRK

SEQ ID NO: 21 - protein, 26 aa, Artificial sequence
  Synthetic penetrating peptide
NYYDITLALAGICQSARLVQQLAGGK

SEQ ID NO: 22 - protein, 30 aa, Artificial sequence
  Synthetic penetrating peptide
NYYDITLALAGICQSARLVQQLAGGGKGGK

SEQ ID NO: 23 - protein, 25 aa, Artificial sequence
  Synthetic penetrating peptide
NYYDITLALAGICQSARLVQQLAGG

SEQ ID NO: 24 - protein, 23 aa, Homo sapiens
NYFLVNLAFAEASMAAFNTVVNF

SEQ ID NO: 25 - protein, 24 aa, Escherichia coli
MNYYDITLALAGICQSARLVQQLA

SEQ ID NO: 26 - protein, 23 aa, Escherichia coli
MYYDITLALAGICQSARLVQQLA

SEQ ID NO: 27 - protein, 22 aa, Escherichia coli
MYDITLALAGICQSARLVQQLA

SEQ ID NO: 28 - protein, 23 aa, Bacillus subtilis
MRNLTRTSLLLAGLCTAAQMVFV

SEQ ID NO: 29 - protein, 23 aa, Haemophilus influenzae
NYHDIVLALAGVCQSARLVHQLA

SEQ ID NO: 30 - protein, 30 aa, Artificial sequence
  Synthetic penetrating peptide
NLPPIVLAVIGICAAVFLLQQYVGGGKGGK

SEQ ID NO: 31 - protein, 30 aa, Artificial sequence
  Synthetic penetrating peptide
NYFIVNLALADLCMAAFNAAFNFGGGKGGK

SEQ ID NO: 32 - protein, 30 aa, Artificial sequence
  Synthetic penetrating peptide
MRNLTRTSLLLAGLCTAAQMVFVGGGKGGK

SEQ ID NO: 33 - protein, 30 aa, Artificial sequence
  Synthetic penetrating peptide
NYHDIVLALAGVCQSARLVHQLAGGKGGKN

SEQ ID NO: 34 - protein, 29 aa, Artificial sequence
  Synthetic penetrating peptide
NYFLVNLAFAEASMAAFNTVVNFGGKGGK

SEQ ID NO: 35 - protein, 31 aa, Artificial sequence
  Synthetic penetrating peptide
MNYYDITLALAGICQSARLVQQLAGGGKGGK

SEQ ID NO: 36 - protein, 30 aa, Artificial sequence
  Synthetic penetrating peptide
MYYDITLALAGICQSARLVQQLAGGGKGGK

SEQ ID NO: 37 - protein, 29 aa, Artificial sequence
  Synthetic penetrating peptide
MYDITLALAGICQSARLVQQLAGGGKGGK

SEQ ID NO: 38 - protein, 16 aa, Artificial sequence
  Synthetic Penetrating peptide consensus sequence
XXXXXXXXXXXXXXXX

SEQ ID NO: 39 - protein, 23 aa, Artificial sequence
  Synthetic Penetrating peptide consensus sequence
XXXXXXXXXXXXXXXXXXXXXXX

SEQ ID NO: 40 - protein, 23 aa, Artificial sequence
  Synthetic Penetrating peptide consensus sequence
XXXXXXXXXXXXXXXXXXXXXXX

SEQ ID NO: 41 - protein, 23 aa, Artificial sequence
  Synthetic Penetrating peptide consensus sequence
XXXXXXXXXXXXXXXXXXXXXXX

SEQ ID NO: 42 - protein, 25 aa, Artificial sequence
  Synthetic Penetrating peptide consensus sequence
XXXXXXXXXXXXXXXXXXXXXXXXX

SEQ ID NO: 43 - protein, 23 aa, Artificial sequence
  Synthetic Penetrating peptide consensus sequence
XXXXXXXXXXXXXXXXXXXXXXX

SEQ ID NO: 44 - protein, 23 aa, Artificial sequence
  Synthetic Penetrating peptide consensus sequence
XXXXXXXXXXXXXXXXXXXXXXX

SEQ ID NO: 45 - protein, 23 aa, Artificial sequence
  Synthetic Penetrating peptide consensus sequence
XXXXXXXXXXXXXXXXXXXXXXX

SEQ ID NO: 46 - protein, 24 aa, Artificial sequence
  Synthetic Penetrating peptide consensus sequence
XXXXXXXXXXXXXXXXXXXXXXXX

SEQ ID NO: 47 - protein, 25 aa, Artificial sequence
  Synthetic Penetrating peptide consensus sequence
XXXXXXXXXXXXXXXXXXXXXXXXX

SEQ ID NO: 48 - protein, 23 aa, Artificial sequence
  Synthetic Penetrating peptide consensus sequence
XXXXXXXXXXXXXXXXXXXXXXX

SEQ ID NO: 49 - protein, 23 aa, Artificial sequence
  Synthetic Penetrating peptide consensus sequence
XXXXXXXXXXXXXXXXXXXXXXX

SEQ ID NO: 50 - protein, 26 aa, Artificial sequence
  Penetrating peptide consensus sequence
XXXXXXXXXXXXXXXXXXXXXXXXXX

SEQ ID NO: 51 - protein, 28 aa, Artificial sequence
  Synthetic Penetrating peptide consensus sequence
XXXXXXXXXXXXXXXXXXXXXXXXXXXX

SEQ ID NO: 52 - protein, 23 aa, Artificial sequence
  Synthetic Penetrating peptide consensus sequence
XXXXXXXXXXXXXXXXXXXXXXX

SEQ ID NO: 53 - protein, 205 aa, Haemophilus influenzae
MKNYHDIVLALAGVCQSAKLVHQLATESRADSETFLTALNSLFITQPQRIEDVFGGEVRHLKLGLETLIHQLNAQGDQNLTRYWLSLLALEGKLSKNSDAKQTLGNRISRLKEQEIHYARDSETMLSIMANIYSDIISPLGKKIHILGSPDYLRQELVQNKIRAVLLAGIRSAVLWKQMGGTKWQILFFRRKLLATAKQIYSSIY

SEQ ID NO: 54 - protein, 203 aa, Pasteurella multocida
MANYYDITLALAGVCQAAKLVQQFAHEGQADQAAFETSLNTLLQIYPEDTLAVFGGKAQNLKLGLETLLEQMHGTGSDLSRYWISLLALESKLNKDPHAKAELARRIQYLPTQLEHYDLLDEQMLSTLASIYVDVISPLGKKIQVTGSTLYLQQLAMHHRIRACLLAGIRSAVLWRQVGGTKWQVLFSRRKIIAMAKQIYSSL

SEQ ID NO: 55 - protein, 213 aa, Escherichia coli
MAKNYYDITLALAGICQSARLVQQLAHQGHCDADALHVSLNSIIDMNPSSTLAVFGGSEANLRVGLETLLGVLNASSRQGLNAELTRYTLSLMVLERKLSSAKGALDTLGNRINGLQRQLEHFDLQSETLMSAMAAIYVDVISPLGPRIQVTGSPAVLQSPQVQAKVRATLLAGIRAAVLWHQVGGGRLQLMFSRNRLTTQAKQILAHLTPEL

SEQ ID NO: 56 - protein, 204 aa, Vibrio cholerae
MANAIYDRTIAFAGICQAVALVQQVAKNGYCDSDAFETSLKAITCTNPSNTLEVFGHESQLKLGLECLVKGIDSTPSGSEITRYLISLMALERKLSGRRDAMSQLGDRIQMIERQLDHFDLFDDQMISNLASIYLDVISPIGPRIQVTGTPAVLQQTANQHKVRALLLSGIRCAVLWRQVGGRRRHLIFGRKKMIEQAQILLAR

SEQ ID NO: 57 - protein, 211 aa, Buchnera aphidicola
MKKIHLITLSLAGICQSAHLVQQLAYSGKCDSNAFSICLKSILEINPTSFIAIYGNHEKNLIIGLEILLSTLTFSSFSYSYIELIKYISNMMIIEKKLKKSRTAIYSLKNKISVISSEYYLNYNIKNLTRKLGELYLEIISSLGSRIVIKGIKDFLQDHQIQEKIRCLLFSGIRAIVLWKQYGGNQLQLIYFRYFIIKKAKKILYHLKDAT

SEQ ID NO: 58 - protein, 206 aa, Pseudomonas aeruginosa
MSDPRQQLIALGAVFESAALVDKLARTGQISEAPLGCMLGSLLARNPASTLDVYGGDSLNLRDGFKALASALERKPGSLQREPLRYALAMLTLERQLDKRGDMLDLIGQRLDQVEQQVQHFGLVHENVIASFASIYQDTLSTFRQRIQVHGDMRHLQVSSNAARIRALLLAGIRSARLWRQLGGSRWQMVFSRRRLLNELYPLLRG

SEQ ID NO: 59 - protein, 204 aa, Xylella fastidiosa
MNALIDNRVLALAGVVQALQQVRQIAETGQSETSAVRTAIDSVLRIDAESPEAVYGGIRDLTQGLQLLHDYFGNQLHDQLLPRLTLAVLQLERRFIRDTSIVAAVSAGITQAAHQVEQTGDSAHPEVLSTLGALYANTISHLRPRIIVQGNPHYLGQAGVVAEIRAMLLAALRSAVLWRQLNGNLLDFLLAKRAMAAATERALR

SEQ ID NO: 60 - protein, 5 aa, Artificial sequence
  Synthetic Fully conserved “strong” amino acid residue chain
NREQK

SEQ ID NO: 61 - protein, 4 aa, Artificial sequence
  Synthetic Fully conserved “strong” amino acid residue chain
NHQK

SEQ ID NO: 62 - protein, 4 aa, Artificial sequence
  Synthetic Fully conserved “strong” amino acid residue chain
NDEQ

SEQ ID NO: 63 - protein, 4 aa, Artificial sequence
  Synthetic Fully conserved “strong” amino acid residue chain
QHRK

SEQ ID NO: 64 - protein, 4 aa, Artificial sequence
  Synthetic Fully conserved “strong” amino acid residue chain
MILV

SEQ ID NO: 65 - protein, 4 aa, Artificial sequence
  Synthetic Fully conserved “strong” amino acid residue chain
MILF

SEQ ID NO: 66 - protein, 4 aa, Artificial sequence
  Synthetic Fully conserved “weak” amino acid residue chain
STNK

SEQ ID NO: 67 - protein, 4 aa, Artificial sequence
  Synthetic Fully conserved “weak” amino acid residue chain
STPA

SEQ ID NO: 68 - protein, 4 aa, Artificial sequence
  Synthetic Fully conserved “weak” amino acid residue chain
SGND

SEQ ID NO: 69 - protein, 6 aa, Artificial sequence
  Synthetic Fully conserved “weak” amino acid residue chain
SNDEQK

SEQ ID NO: 70 - protein, 6 aa, Artificial sequence
  Synthetic Fully conserved “weak” amino acid residue chain
NDEQHK

SEQ ID NO: 71 - protein, 6 aa, Artificial sequence
  Synthetic Fully conserved “weak” amino acid residue chain
NEQHRK

SEQ ID NO: 72 - protein, 23 aa, Bacillus subtilis
NLTKTSLLLAGLCTAAQMVFVTH